(19) United States
(12) Patent Application Publication
(10) Pub. No.: US 2011/0003859 A1
(43) Pub. Date: Jan. 6, 2011

(54) N- (6-AMINOPYRIDIN-3-YL) -3- (SULFONAMIDO) BENZAMIDE DERIVATIVES AS B-RAF INHIBITORS FOR THE TREATMENT OF CANCER

(75) Inventor: Kateri A. Ahrendt, Boulder, CO (US)

Correspondence Address: VIKSNINS HARRIS & PADYS PLLP, P.O. BOX 111098, ST. PAUL, MN, 55111-1098, US

(73) Assignee: Array Biopharma Inc., Boulder, CO (US)

(21) Appl. No.: 12/919,972

(22) PCT Filed: Feb. 27, 2009
(86) PCT No.: PCT/US09/35383

§ 371 Date: Aug. 27, 2010

Related U.S. Application Data
(60) Provisional application No. 61/032,795, filed on Feb. 29, 2008.

(51) Int. Cl. A61K 31/444; C07D 213/02; C07D 401/04; C07D 213/04; C07D 417/04; C07D 405/04; A61K 31/4439; A61K 31/443; A61K 31/4427; A61K 31/44; A61P 35/00; A61P 35/02

(52) U.S. Cl. 514/334; 546/308; 546/275.4; 546/265; 546/268.1; 546/270.4; 546/283.4; 514/340; 514/341; 514/342; 514/352

(57) ABSTRACT

Compounds of Formula (I) are useful for inhibition of Raf kinases. Methods of using compounds of Formula I and stereoisomers and pharmaceutically acceptable salts thereof, for in vitro, in situ, and in vivo diagnosis, prevention or treatment of such disorders in mammalian cells, or associated pathological conditions are disclosed.

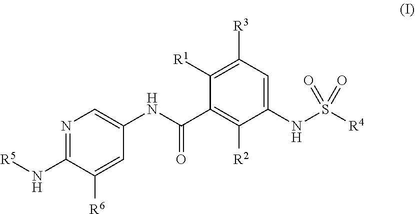

### BACKGROUND OF THE INVENTION

1. Field of the Invention

The present invention relates to novel compounds, to pharmaceutical compositions comprising the compounds, to a process for making the compounds and to the use of the compounds in therapy. More particularly, it relates to certain substituted 6-aminopyridine compounds useful for inhibiting Raf kinase and for treating disorders mediated thereby.

2. Description of the State of the Art

The Raf/MEK/ERK pathway is critical for cell survival, growth, proliferation and tumorigenesis. Li, Nanxin, et al. “B-Raf kinase inhibitors for cancer treatment.” Current Opinion in Investigational Drugs. Vol. 8, No. 6 (2007): 452-456. Raf kinases exist as three isoforms, A-Raf, B-Raf and C-Raf. Among the three isoforms, studies have shown that B-Raf functions as the primary MEK activator. B-Raf is one of the most frequently mutated genes in human cancers. B-Raf kinase represents an excellent target for anticancer therapy based on preclinical target validation, epidemiology and drugability.

Small molecule inhibitors of B-Raf are being developed for anticancer therapy. Nexavar® (sorafenib tosylate) is a multikinase inhibitor, which includes inhibition of B-Raf, and is approved for the treatment of patients with advanced renal cell carcinoma and unresectable hepatocellular carcinoma. Other Raf inhibitors have also been disclosed or have entered clinical trials, for example SB-590885, RAF-265, PLX-4032 and XL-281. Other B-Raf inhibitors are also known, see for example, U.S. Patent Application Publication 2006/0189627, U.S. Patent Application Publication 2006/0281751, U.S. Patent Application Publication 2007/0049603, International Patent Application Publication WO 2007/002325 and International Patent Application Publication WO 2007/002433.

Aminopyridines are known, see for example, International Patent Application Publication WO 2006/067445 and International Patent Application Publication WO 2006/067446.

Kinase inhibitors are known, see for example, International Patent Application Publication WO 2005/062795 and International Patent Application Publication WO 2007/013896.

International Patent Application Publication WO 2006/066913, International Patent Application Publication WO 2008/028617 and International Patent Application Publication WO 2009/012283 also disclose kinase inhibitors.

### SUMMARY OF THE INVENTION

In one aspect, the invention relates to compounds that are inhibitors of Raf kinases, particularly B-Raf inhibitors. Certain hyperproliferative disorders are characterized by the over activation of Raf kinase function, for example by mutations or over expression of the protein. Accordingly, the compounds of the invention are useful in the treatment of hyperproliferative disorders such as cancer.

More specifically, one aspect of the present invention provides compounds of Formula I:

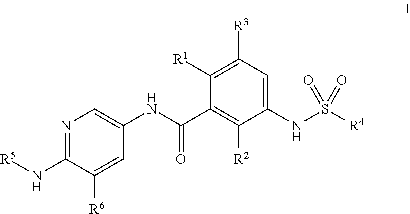

and stereoisomers, tautomers and pharmaceutically acceptable salts thereof, wherein R¹, R², R³, R⁴, R⁵ and R⁶ are as defined herein.

Another aspect of the present invention provides methods of preventing or treating a disease or disorder modulated by B-Raf, comprising administering to a mammal in need of such treatment an effective amount of a compound of this invention or a stereoisomer or pharmaceutically acceptable salt thereof. Examples of such diseases and disorders include, but are not limited to, hyperproliferative disorders (such as cancer, including melanoma and other cancers of the skin), neurodegeneration, cardiac hypertrophy, pain, migraine and neurotraumatic disease.

Another aspect of the present invention provides methods of preventing or treating cancer, comprising administering to a mammal in need of such treatment an effective amount of a compound of this invention, or a stereoisomer or pharmaceutically acceptable salt thereof, alone or in combination with one or more additional compounds having anti-cancer properties.

Another aspect of the present invention provides a method of treating a hyperproliferative disease in a mammal comprising administering a therapeutically effective amount of a compound of this invention to the mammal.

Another aspect of the present invention provides methods of preventing or treating kidney disease, comprising administering to a mammal in need of such treatment an effective amount of a compound of this invention, or a stereoisomer or pharmaceutically acceptable salt thereof, alone or in combination with one or more additional compounds. Another aspect of the present invention provides methods of preventing or treating polycystic kidney disease, comprising administering to a mammal in need of such treatment an effective amount of a compound of this invention, or a stereoisomer or pharmaceutically acceptable salt thereof, alone or in combination with one or more additional compounds.

Another aspect of the present invention provides the compounds of the present invention for use in therapy.

Another aspect of the present invention provides the compounds of the present invention for use in the treatment of a hyperproliferative disease. In a further embodiment, the hyperproliferative disease may be cancer (or still further, a specific cancer as defined herein).

Another aspect of the present invention provides the compounds of the present invention for use in the treatment of a kidney disease. In a further embodiment, the kidney disease may be polycystic kidney disease.

Another aspect of the present invention provides the use of a compound of this invention in the manufacture of a medicament for the treatment of a hyperproliferative disease. In a further embodiment, the hyperproliferative disease may be cancer (or still further, a specific cancer as defined herein).

Another aspect of the present invention provides the use of a compound of this invention in the manufacture of a medicament for the treatment of a kidney disease. In a further embodiment, the kidney disease may be polycystic kidney disease.

Another aspect of the present invention provides the use of a compound of the present invention in the manufacture of a medicament, for use as a B-Raf inhibitor in the treatment of a patient undergoing cancer therapy.

Another aspect of the present invention provides the use of a compound of the present invention in the manufacture of a medicament, for use as a B-Raf inhibitor in the treatment of a patient undergoing polycystic kidney disease therapy.

Another aspect of the present invention provides a pharmaceutical composition comprising a compound of the present invention for use in the treatment of a hyperproliferative disease.

Another aspect of the present invention provides a pharmaceutical composition comprising a compound of the present invention for use in the treatment of cancer.

Another aspect of the present invention provides a pharmaceutical composition comprising a compound of the present invention for use in the treatment of polycystic kidney disease.

Another aspect of the present invention provides a pharmaceutical composition comprising a compound of this invention or a pharmaceutically acceptable salt thereof, and a pharmaceutically acceptable carrier or excipient.

Another aspect of the present invention provides intermediates for preparing compounds of Formula I. Certain compounds of Formula I may be used as intermediates for other compounds of Formula I.

Another aspect of the present invention includes methods of preparing, methods of separation, and methods of purification of the compounds of this invention.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “alkyl” includes linear or branched-chain radicals of carbon atoms. In one example, the alkyl radical is one to six carbon atoms (C₁-C₆). In other examples, the alkyl radical is C₁-C₅, C₁-C₄ or C₁-C₃. Some alkyl moieties have been abbreviated, for example, methyl (“Me”), ethyl (“Et”), propyl (“Pr”) and butyl (“Bu”), and further abbreviations are used to designate specific isomers of compounds, for example, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”) and the like. Other examples of alkyl groups include 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂) and 3,3-dimethyl-2-butyl (—CH(CH₃)C(CH₃)₃. The abbreviations are sometimes used in conjunction with elemental abbreviations and chemical structures, for example, methanol (“MeOH”) or ethanol (“EtOH”).

Additional abbreviations used throughout the application include, for example, benzyl (“Bn”), phenyl (“Ph”) and acetyl (“Ac”).

The term “alkenyl” refers to linear or branched-chain monovalent hydrocarbon radical with at least one site of unsaturation, i.e., a carbon-carbon double bond, wherein the alkenyl radical may be optionally substituted independently with one or more substituents described herein, and includes radicals having “cis” and “trans” orientations, or alternatively, “E” and “Z” orientations. In one example, the alkenyl radical is two to six carbon atoms (C₂-C₆). In other examples, the alkenyl radical is C₂-C₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, hexa-1,3-dienyl.

The term “alkynyl” refers to a linear or branched monovalent hydrocarbon radical with at least one site of unsaturation, i.e., a carbon-carbon, triple bond, wherein the alkynyl radical may be optionally substituted independently with one or more substituents described herein. In one example, the alkynyl radical is two to eighteen carbon atoms (C₂-C₆). In other examples, the alkynyl radical is C₂-C₃. Examples include, but are not limited to, ethynyl (—C≡CH), prop-1-ynyl (—C≡CCH₃), prop-2-ynyl (propargyl, CH₂C≡CH), but-1-ynyl, but-2-ynyl and but-3-ynyl.

The terms “alkenyl” and “alkynyl” also include linear or branched-chain radicals of carbon atoms containing at least one unsaturated bond.

“Cycloalkyl” refers to a non-aromatic, saturated or partially unsaturated hydrocarbon ring group wherein the cycloalkyl group may be optionally substituted independently with one or more substituents described herein. In one example, the cycloalkyl group is 3 to 6 carbon atoms (C₃-C₆). In other examples, cycloalkyl is C₃-C₄ or C₃-C₅. In other examples, the cycloalkyl group, as a monocycle, is C₃-C₆ or C₅-C₆. In another example, the cycloalkyl group, as a bicycle, is C₇-C₁₂. Examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane, and bicyclo[3.2.2]nonane.

The terms “heterocyclic” or “heterocycle” or “heterocyclyl” refers to a saturated or a partially unsaturated (i.e., having one or more double and/or triple bonds within the ring) cyclic group in which at least one ring atom is a heteroatom independently selected from nitrogen, oxygen, and sulfur, the remaining ring atoms being carbon. In one embodiment, heterocyclyl includes saturated or partially unsaturated 4-6 membered heterocyclyl groups. The heterocyclyl group may be optionally substituted with one or more substituents described herein. Exemplary heterocyclyl groups include, but are not limited to, oxiranyl, aziridinyl, thiiranyl, azetidinyl, oxetanyl, thietanyl, 1,2-dithietanyl, 1,3-dithietanyl, pyrrolidinyl, piperidinyl, dihydropyridinyl, tetrahydropyridinyl, morpholinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepam 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, imidazolidinyl, pyrimidinonyl, 1,1-dioxo-thiomorpholinyl, 3-azabicyco [3.1.0]hexanyl, 3-azabicyclo[4.1.0]heptanyl and azabicyclo[2.2.2]hexanyl. Heterocycles include 4 to 6 membered rings containing one or two heteroatoms selected from oxygen, nitrogen and sulfur.

The term “heteroaryl” refers to an aromatic cyclic group in which at least one ring atom is a heteroatom independently selected from nitrogen, oxygen and sulfur, the remaining ring atoms being carbon. Heteroaryl groups may be optionally substituted with one or more substituents described herein. In one example, heteroaryl includes 5-6 membered heteroaryl groups. Other examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, and furopyridinyl. Heteroaryls include 5 to 6 membered aromatic rings containing one, two or three heteroatoms selected from oxygen, nitrogen and sulfur.

“Halogen” refers to F, Cl, Br or I.

The terms “treat” or “treatment” refer to therapeutic, prophylactic, palliative or preventative measures. In one example, treatment includes therapeutic and palliative treatment. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrases “therapeutically effective amount” or “effective amount” mean an amount of a compound of the present invention that, when administered to a mammal in need of such treatment, sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the mammal in need of treatment, but can nevertheless be routinely determined by one skilled in the art.

The terms “cancer” and “cancerous” refer to or describe the physiological condition in mammals that is typically characterized by abnormal or unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term cancer may be used generically to include various types of cancer or specifically (as listed above).

The phrase “pharmaceutically acceptable” indicates that the substance or composition is compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound of the invention.

The compounds of this invention also include other salts of such compounds which are not necessarily pharmaceutically acceptable salts, and which may be useful as intermediates for preparing and/or purifying compounds of this invention and/or for separating enantiomers of compounds of this invention.

The term “mammal” means a warm-blooded animal that has or is at risk of developing a disease described herein and includes, but is not limited to, guinea pigs, dogs, cats, rats, mice, hamsters, and primates, including humans.

### B-Raf Inhibitor Compounds

The present invention provides compounds, and pharmaceutical formulations thereof, that are potentially useful in the treatment of diseases, conditions and/or disorders modulated by B-Raf.

One embodiment of this invention provides compounds of Formula I:

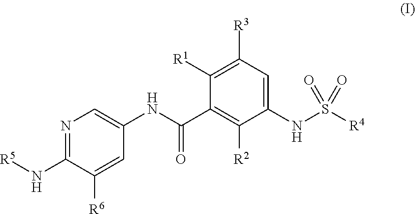

and stereoisomers, tautomers and pharmaceutically acceptable salts thereof, wherein:

R¹ and R² are independently selected from hydrogen, halogen, CN, C₁-C₃ alkyl and C₁-C₃ alkoxy;

R⁴ is C₃-C₅ cycloalkyl, C₁-C₆ alkyl, C₂-C₆ alkenyl, C₂-C₆ alkynyl, phenyl, a 5-6 membered heteroaryl, or NR^{b}R^{c}, wherein the cycloalkyl, alkyl, alkenyl, alkynyl and phenyl are optionally substituted with OR^{a}, halogen, phenyl, C₃-C₄ cycloalkyl or C₁-C₄ alkyl optionally substituted with halogen;

R⁵ is hydrogen, —C(═O)(C₁-C₄ alkyl), phenyl optionally substituted with halogen or C₁-C₄ alkyl, or a 5-6 membered heteroaryl;

R⁶ is hydrogen, halogen, CN, —SO₂(C₁-C₄ alkyl), C₁-C₄ alkyl, —C(═O)R^{d} or a 5-6 membered heteroaryl optionally substituted with C₁-C₄ alkyl;

each R^{a} is hydrogen or C₁-C₄ alkyl;

each R^{b} and R^{c} are independently selected from hydrogen and C₁-C₅ alkyl optionally substituted with halogen, or

R^{b} and R^{c} together with the nitrogen to which they are attached form a 4 to 6 membered heterocyclic ring;

R^{d} is —O(C₁-C₆ alkyl), NR^{e}R^{f} or a 4 membered heterocycle; and

each R^{e} and R^{f} are independently selected from hydrogen and C₁-C₆ alkyl.

One embodiment of this invention provides compounds of Formula I:

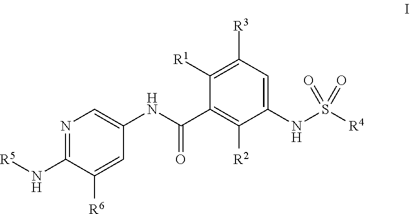

and stereoisomers, tautomers and pharmaceutically acceptable salts thereof, wherein:

R¹ and R² are independently selected from hydrogen, halogen, CN, C₁-C₃ alkyl and C₁-C₃ alkoxy;

R⁴ is C₃-C₅ cycloalkyl, C₁-C₆ alkyl, C₂-C₆ alkenyl, or C₂-C₆ alkynyl, wherein the cycloalkyl, alkyl, alkenyl and alkynyl are optionally substituted with halogen, OR^{a} or C₃-C₄ cycloalkyl;

R⁵ is hydrogen, —C(═O)(C₁-C₄ alkyl), phenyl optionally substituted with halogen or C₁-C₄ alkyl, or a 5-6 membered heteroaryl;

R⁶ is hydrogen, halogen, CN, —SO₂(C₁-C₄ alkyl), C₁-C₄ alkyl, or a 5-6 membered heteroaryl optionally substituted with C₁-C₄ alkyl; and

each R^{a} is hydrogen or C₁-C₄ alkyl.

Compounds of Formula I include compounds wherein:

R¹, R² and R³ are independently selected from hydrogen, halogen or C₁-C₃ alkyl;

R⁴ is C₃-C₄ cycloalkyl, or C₁-C₆ alkyl optionally substituted with halogen, OH or C₃-C₄ cycloalkyl;

R⁵ is hydrogen, —C(═O)(C₁-C₄ alkyl), phenyl optionally substituted with halogen or C₁-C₄ alkyl, or a 5-6 membered heteroaryl; and

R⁶ is hydrogen, halogen, CN, —SO₂(C₁-C₄ alkyl), C₁-C₄ alkyl, or a 5-6 membered heteroaryl optionally substituted with C₁-C₄ alkyl.

In certain embodiments, R¹ and R² are independently selected from hydrogen, halogen, CN, C₁-C₃ alkyl or C₁-C₃ alkoxy.

In certain embodiments, R¹, R² and R³ are independently selected from hydrogen, halogen or C₁-C₃ alkyl.

In certain embodiments, R¹, R² and R³ are independently selected from hydrogen, F and Cl.

In certain embodiments, R¹ is hydrogen, halogen, CN, C₁-C₃ alkyl or C₁-C₃ alkoxy.

In certain embodiments, R¹ is hydrogen.

In certain embodiments, R¹ is halogen. In certain embodiments, R¹ is F or Cl.

In certain embodiments, R¹ is C₁-C₃ alkyl. In certain embodiments, R¹ is methyl.

In certain embodiments, R² is hydrogen, halogen, CN, C₁-C₃ alkyl or C₁-C₃ alkoxy.

In certain embodiments, R² is hydrogen.

In certain embodiments, R² is halogen. In certain embodiments, R² is F or Cl.

In certain embodiments, R² is C₁-C₃ alkyl. In certain embodiments, R² is methyl.

In certain embodiments of Formula I, R² is Cl.

In certain embodiments of Formula I, R² is hydrogen.

In certain embodiments, R³ is hydrogen, halogen or C₁-C₃ alkyl.

In certain embodiments, R³ is hydrogen.

In certain embodiments, R³ is halogen. In certain embodiments, R³ is F or Cl.

In certain embodiments, R¹ and R² are F and R³ is hydrogen.

In certain embodiments, R¹ is F, R² is Cl and R³ is hydrogen.

In certain embodiments, R¹ is Cl, R² is F and R³ is hydrogen.

In certain embodiments, R¹ is F and R² and R³ are hydrogen.

In certain embodiments, R¹ and R³ are hydrogen and R² is F.

In certain embodiments, R² and R³ are F and R¹ is hydrogen.

In certain embodiments, R¹ is Cl, R² and R³ are hydrogen.

In certain embodiments, R¹, R² and R³ are F.

In certain embodiments, R¹ is F and R² is methyl and R³ is hydrogen.

In certain embodiments, R¹ is methyl and R² is F and R³ is hydrogen.

In certain embodiments, R¹ is F and R² and R³ are hydrogen.

In certain embodiments, R¹ is Cl and R² and R³ are hydrogen.

In certain embodiments, R² is F and R¹ and R³ are hydrogen.

In certain embodiments, the residue:

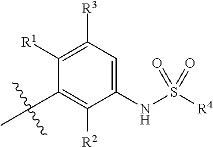

of Formula I, wherein the wavy line represents the point of attachment of the residue in Formula I, is selected from:

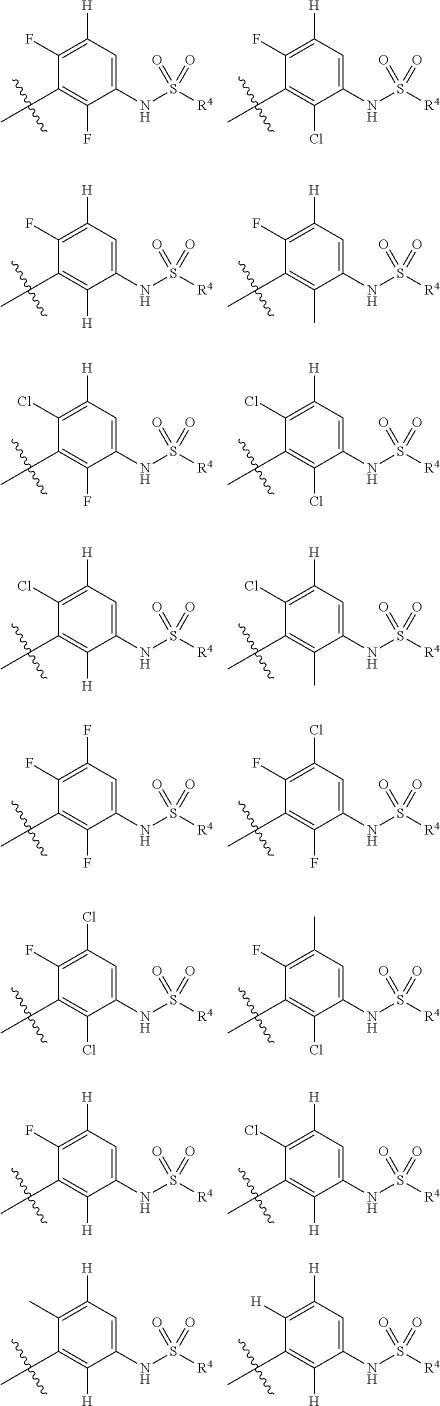

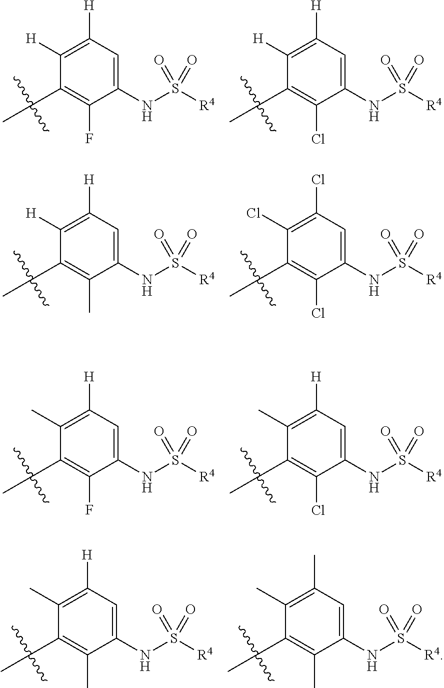

In certain embodiments, R⁴ is C₃-C₅ cycloalkyl, C₁-C₆ alkyl, C₂-C₆ alkenyl, C₂-C₆ alkynyl, phenyl, a 5-6 membered heteroaryl, or NR^{b}R^{c}, wherein the cycloalkyl, alkyl, alkenyl, alkynyl and phenyl are optionally substituted with OR^{a}, halogen, phenyl, C₃-C₄ cycloalkyl or C₁-C₄ alkyl optionally substituted with halogen.

In certain embodiments, R^{a} is independently selected from hydrogen, phenyl and C₁-C₄ alkyl optionally substituted with oxo. In certain embodiments, R^{a} is hydrogen.

In certain embodiments, R⁴ is C₃-C₅ cycloalkyl, C₁-C₆ alkyl, C₂-C₆ alkenyl, C₂-C₆ alkynyl, phenyl, a 5-6 membered heteroaryl, or NR^{b}R^{c}, wherein the cycloalkyl, alkyl, alkenyl, alkynyl and phenyl are optionally substituted with OH, halogen, phenyl, C₃-C₄ cycloalkyl or C₁-C₄ alkyl optionally substituted with halogen.

In certain embodiments, R⁴ is C₃-C₅ cycloalkyl, C₁-C₆ alkyl, C₂-C₆ alkenyl, or C₂-C₆ alkynyl, wherein the cycloalkyl, alkyl, alkenyl and alkynyl are optionally substituted with OR^{a}, halogen or C₃-C₄ cycloalkyl.

In certain embodiments, R⁴ is cyclopropyl, ethyl, propyl, butyl, isobutyl, —CH₂CH₂CH₂OH, —CH₂Cl, —CH₂CF₃, —CH₂CH₂CH₂F, —CH₂CH₂CF₃, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl, —NHCH₂CH₃, —NHCH₂CH₂CH₃, —N(CH₃)CH₂CH₃, —NHCH(CH₃)₂, —NHCH₂CHF₂, —N(CH₃)₂ or pyrrolidin-1-yl.

In certain embodiments, R⁴ is cyclopropyl, ethyl, propyl, isobutyl, —CH₂CH₂CH₂OH, —CH₂CH₂CH₂F, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, thiophen-2-yl or —NHCH₂CH₃.

In certain embodiments, R⁴ is propyl, butyl, isobutyl, —CH₂CH₂CH₂F, —CH₂CH₂CF₃ or cyclopropylmethyl.

In certain embodiments, R⁴ is C₃-C₅ cycloalkyl or C₁-C₆ alkyl optionally substituted with halogen, OH or C₃-C₄ cycloalkyl.

In certain embodiments, R⁴ is C₃-C₅ cycloalkyl. In certain embodiments, R⁴ is C₃-C₄ cycloalkyl. In certain embodiments, R⁴ is cyclopropyl or cyclobutyl.

In certain embodiments, R⁴ is C₃-C₅ cycloalkyl. In certain embodiments, R⁴ is C₃-C₄ cycloalkyl. In certain embodiments, R⁴ is cyclopropyl.

In certain embodiments, R⁴ is C₁-C₆ alkyl. In certain embodiments, R⁴ is ethyl, propyl, butyl or isobutyl.

In certain embodiments, R⁴ is C₁-C₆ alkyl. In certain embodiments, R⁴ is propyl, butyl or isobutyl.

In certain embodiments, R⁴ is C₁-C₆ alkyl optionally substituted with OR^{a}. In certain embodiments, R^{a} is hydrogen. In certain embodiments, R⁴ is C₁-C₆ alkyl optionally substituted with OH. In certain embodiments, R⁴ is —CH₂CH₂CH₂OH.

In certain embodiments, R⁴ is C₁-C₆ alkyl optionally substituted with halogen. In certain embodiments, R⁴ is —CF₃, —CH₂Cl, —CH₂CF₃, —CH₂CH₂CH₂F, —CH₂CH₂CF₃, —CF₂CF₃ or —CF₂CF₂CF₃.

In certain embodiments, R⁴ is C₁-C₆ alkyl optionally substituted with halogen. In certain embodiments, R⁴ is —CF₃, —CH₂CF₃, —CH₂CH₂CH₂F, —CH₂CH₂CF₃, —CF₂CF₃ or —CF₂CF₂CF₃. In certain embodiments, R⁴ is —CH₂CH₂CH₂F or —CH₂CH₂CF₃.

In certain embodiments, R⁴ is C₁-C₆ alkyl substituted with halogen, OR^{a} or C₃-C₄ cycloalkyl. In certain embodiments, R⁴ is C₁-C₆ alkyl substituted with halogen, OH or C₃-C₄ cycloalkyl. In certain embodiments, R⁴ is cyclopropylmethyl (—CH₂-cyclopropyl) or cyclobutylmethyl (—CH₂-cyclobutyl). In certain embodiments, R⁴ is cyclopropylmethyl (—CH₂-cyclopropyl).

In certain embodiments, R⁴ is C₁-C₆ alkyl optionally substituted with phenyl. In certain embodiments, R⁴ is phenylmethyl.

In certain embodiments, R⁴ is phenyl optionally substituted with OR^{a}, halogen, C₃-C₄ cycloalkyl or C₁-C₄ alkyl optionally substituted with halogen. In certain embodiments, R⁴ is phenyl optionally substituted with halogen. In certain embodiments, R⁴ is phenyl optionally substituted with C₁-C₄ alkyl optionally substituted with halogen. In certain embodiments, R⁴ is phenyl optionally substituted with halogen and C₁-C₄ alkyl optionally substituted with halogen. In certain embodiments, R⁴ is phenyl. In certain embodiments, R⁴ is phenyl, 2-fluorophenyl, 3-fluorophenyl, 4-fluorophenyl, 2,5-difluorophenyl or 4-chloro-3-trifluoromethylphenyl.

In certain embodiments, R⁴ is a 5-6 membered heteroaryl optionally substituted with OR^{a}, halogen, C₃-C₄ cycloalkyl or C₁-C₄ alkyl optionally substituted with halogen. In certain embodiments, R⁴ is a 5-6 membered heteroaryl optionally substituted with C₁-C₄ alkyl. In certain embodiments, R⁴ is a 5-6 membered heteroaryl optionally substituted with OR^{a}, halogen, C₃-C₄ cycloalkyl or C₁-C₄ alkyl optionally substituted with halogen, wherein the heteroaryl contains one or two heteroatoms selected from the group consisting of oxygen, nitrogen and sulfur. In certain embodiments, R⁴ is a 5-6 membered heteroaryl optionally substituted with OR^{a}, halogen, C₃-C₄ cycloalkyl or C₁-C₄ alkyl optionally substituted with halogen, wherein the heteroaryl is imidazolyl, furanyl, pyridinyl or thiophenyl. In certain embodiments, R⁴ is 1-methyl-1H-imidazol-4-yl, furan-2-yl, pyridin-2-yl, pyridin-3-yl or thiophen-2-yl.

In certain embodiments, R⁴ is NR^{b}R^{c}. In certain embodiments, R^{b} and R^{c} are independently selected from hydrogen and C₁-C₅ alkyl optionally substituted with halogen. In certain embodiments, R^{c} is hydrogen or methyl. In certain embodiments, R^{b} is C₁-C₅ alkyl optionally substituted with halogen. In certain embodiments, R^{b} is methyl, ethyl, propyl, isopropyl, or 2,2-difluoroethyl. In certain embodiments, R⁴ is selected from the group consisting of —NHCH₂CH₃, —NHCH₂CH₂CH₃, —N(CH₃)CH₂CH₃, —NHCH(CH₃)₂, —NHCH₂CHF₂, and —N(CH₃)₂.

In certain embodiments, R⁴ is NR^{b}R^{c}, wherein R^{b} and R^{c} together with the nitrogen to which they are attached form a 4 to 6 membered heterocyclic ring. In certain embodiments, R⁴ is NR^{b}R^{c}, wherein R^{b} and R^{c} together with the nitrogen to which they are attached form a 4 to 6 membered heterocyclic ring, wherein the heterocyclic ring contains one or two heteroatoms selected from nitrogen and oxygen. In certain embodiments, R⁴ is NR^{b}R^{c}, wherein R^{b} and R^{c} together with the nitrogen to which they are attached form a 5 membered heterocyclic ring. In certain embodiments, R⁴ is NR^{b}R^{c}, wherein R^{b} and R^{c} together with the nitrogen to which they are attached form a 5 membered heterocyclic ring, wherein the heterocyclic ring contains one nitrogen heteroatom. In certain embodiments, R⁴ is pyrrolidin-1-yl.

In certain embodiments, R¹ and R² are F, R³ is hydrogen and R⁴ is propyl, such that the compounds of Formula I, have the structure of Formula Ia:

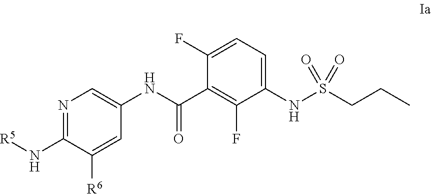

wherein R⁵ and R⁶ are as defined herein.

In certain embodiments, R¹ is Cl, R² is F, R³ is hydrogen and R⁴ is propyl, such that the compounds of Formula I, have the structure of Formula Ia1:

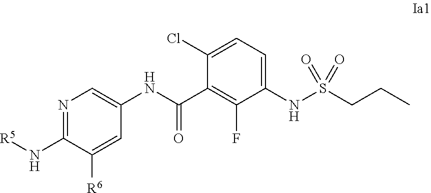

wherein R⁵ and R⁶ are as defined herein.

In certain embodiments, R¹ is F, R² is Cl, R³ is hydrogen and R⁴ is propyl, such that the compounds of Formula I, have the structure of Formula Ia2:

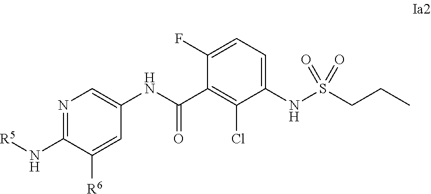

wherein R⁵ and R⁶ are as defined herein.

In certain embodiments, R⁶ is hydrogen, halogen, CN, —SO₂(C₁-C₄ alkyl), C₁-C₄ alkyl, —C(═O)R^{d} or a 5-6 membered heteroaryl optionally substituted with C₁-C₄ alkyl.

In certain embodiments, R^{d} is —O(C₁-C₆ alkyl), NR^{e}R^{f} or a 4 membered heterocycle. In certain embodiments, each R^{e} and R^{f} are independently selected from hydrogen and C₁-C₆ alkyl.

In certain embodiments, R⁶ is hydrogen, halogen, CN, —SO₂(C₁-C₄ alkyl), C₁-C₄ alkyl, or a 5-6 membered heteroaryl optionally substituted with C₁-C₄ alkyl.

In certain embodiments, R⁶ is selected from hydrogen, halogen, CN, —SO₂CH₃, methyl, —C(═O)CH₂CH₃, —C(═O)(azetidin-1-yl), —C(═O)NH₂, —C(═O)NHCH₃, pyridin-3-yl, pyridin-2-yl, 1-methyl-1H-pyrazol-4-yl, furan-2-yl and thiazol-2-yl.

In certain embodiments, R⁶ is selected from hydrogen, halogen, CN, —SO₂CH₃, methyl, pyridin-3-yl and 1-methyl-1H-pyrazol-4-yl.

In certain embodiments, R⁶ is hydrogen.

In certain embodiments, R⁶ is halogen. In certain embodiments, R⁶ is Br.

In certain embodiments, R⁶ is CN.

In certain embodiments, R⁶ is —SO₂(C₁-C₄ alkyl). In certain embodiments, R⁶ is —SO₂CH₃.

In certain embodiments, R⁶ is C₁-C₄ alkyl. In certain embodiments, R⁶ is methyl.

In certain embodiments, R⁶ is —C(═O)R^{d}. In certain embodiments, R^{d} is —O(C₁-C₆ alkyl), NR^{e}R^{f} or a 4 membered heterocycle. In certain embodiments, R^{d} is —O(C₁-C₆ alkyl). In certain embodiments, R^{d} is NR^{e}R^{f}. In certain embodiments, each R^{e} and R^{f} are independently selected from hydrogen and C₁-C₆ alkyl. In certain embodiments, R^{d} is 4 membered heterocycle, wherein the heterocycle contains one or two heteroatoms selected from nitrogen, oxygen and sulfur. In certain embodiments, R^{d} is 4 membered heterocycle, wherein the heterocycle contains one nitrogen heteroatom. In certain embodiments, R⁶ is —C(═O)CH₂CH₃, —C(═O)(azetidin-1-yl), —C(═O)NH₂ or —C(═O)NHCH₃.

In certain embodiments, R⁶ is a 5-6 membered heteroaryl optionally substituted with C₁-C₄ alkyl. In certain embodiments, R⁶ is a 5-6 membered heteroaryl, wherein the heteroaryl is pryidinyl or pyrazolyl. In certain embodiments, R⁶ is pyridin-3-yl or 1-methyl-1H-pyrazol-4-yl.

In certain embodiments, R⁶ is a 5-6 membered heteroaryl optionally substituted with C₁-C₄ alkyl. In certain embodiments, R⁶ is a 5-6 membered heteroaryl optionally substituted with C₁-C₄ alkyl, wherein the heteroaryl contains one, two or three heteroatoms selected from nitrogen, oxygen and sulfur. In certain embodiments, R⁶ is a 5-6 membered heteroaryl optionally substituted with C₁-C₄ alkyl, wherein the heteroaryl contains one or two heteroatoms selected from nitrogen, oxygen and sulfur. In certain embodiments, R⁶ is a 5-6 membered heteroaryl, wherein the heteroaryl is pyridinyl, pyrazolyl, furanyl or thiazolyl. In certain embodiments, R⁶ is pyridin-3-yl, pyridin-2-yl, 1-methyl-1H-pyrazol-4-yl, furan-2-yl or thiazol-2-yl.

In certain embodiments, R⁵ is hydrogen, —C(═O)(C₁-C₄ alkyl), phenyl optionally substituted with halogen or C₁-C₄ alkyl, or a 5-6 membered heteroaryl. In certain embodiments, R⁵ is selected from hydrogen, —C(═O)CH₃, phenyl, 4-fluorophenyl and pyridin-2-yl.

In certain embodiments, R⁵ is hydrogen.

In certain embodiments, R⁵ is —C(═O)(C₁-C₄ alkyl). In certain embodiments, R⁵ is —C(═O)CH₃.

In certain embodiments, R⁵ is phenyl optionally substituted with halogen or C₁-C₄ alkyl. In certain embodiments, R⁵ is phenyl or 4-fluorophenyl.

In certain embodiments, R⁵ is a 5-6 membered heteroaryl. In certain embodiments, R⁵ is a 5-6 membered heteroaryl, wherein the heteroaryl is pyridinyl. In certain embodiments, R⁵ is pyridin-2-yl.

It will be appreciated that certain compounds of the invention may contain asymmetric or chiral centers, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

It will also be appreciated that compounds of Formula I include tautomeric forms. Tautomers are compounds that are interconvertible by tautomerization. This commonly occurs due to the migration of a hydrogen atom or proton, accompanied by the switch of a single bond and adjacent double bond. Tautomers of Formula I may form at the sulfonamide or other positions depending on the substitutions. The compounds of Formula I are intended to include all tautomeric forms.

In another embodiment of the present invention, intermediates of Formula III are provided:

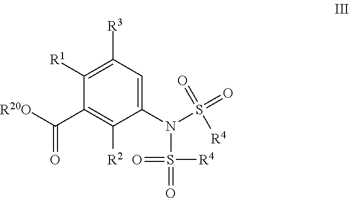

wherein R²⁰ is hydrogen, C₁-C₆ alkyl, benzyl or phenyl and R¹, R², R³ and R⁴ are as defined herein.

It will also be appreciated that certain compounds of Formula I may be used as intermediates for further compounds of Formula I.

It will be further appreciated that the compounds of the present invention may exist in unsolvated, as well as solvated forms with pharmaceutically acceptable solvents such as water, ethanol, and the like, and it is intended that the invention embrace both solvated and unsolvated forms.

### Synthesis of Compounds

Compounds of the present invention may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis. v. 1-23, New York: Wiley 1967-2006 ed. (also available via the Wiley InterScience® website), or Beilsteins Handbuch der organischen Chemie, 4, Aufl. ed. Springer-Verlag, Berlin, including supplements (also available via the Beilstein online database)).

For illustrative purposes, Schemes 1-7 shows a general method for preparing the compounds of the present invention as well as key intermediates. For a more detailed description of the individual reaction steps, see the Examples section below. Those skilled in the art will appreciate that other synthetic routes may be used to synthesize the inventive compounds. Although specific starting materials and reagents are depicted in the Schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

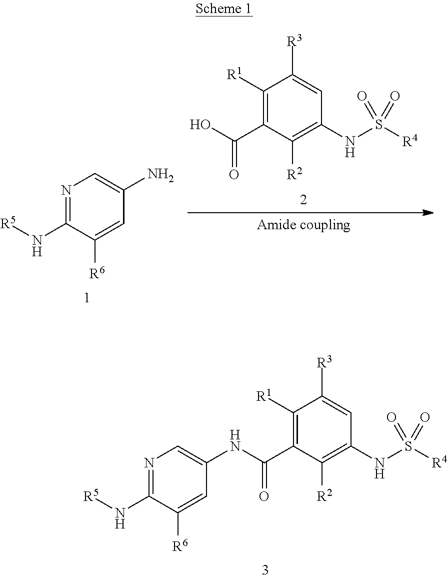

Scheme 1 shows a general scheme for the synthesis of 6-aminopyridin-3-ylbenzamides 3, wherein R¹, R², R³, R⁴, R⁵ and R⁶ are as defined herein. Substituted 6-aminopyridine 1 may be coupled with benzoic acid 2 in the presence of a coupling reagent, such as 2-(1H-benzotriazole-1-yl)-1,1,3,3-tetramethyluronium hexafluorophosphate (“HBTU”), or 1-ethyl-(3-dimethylaminopropyl)carbodiimide hydrochloride (“EDCl”), with additives, such as hydroxybenzotriazole monohydrate, in a suitable solvent, such as dichloromethane (“DCM”), N,N-dimethylformamide (“DMF”) or mixtures thereof.

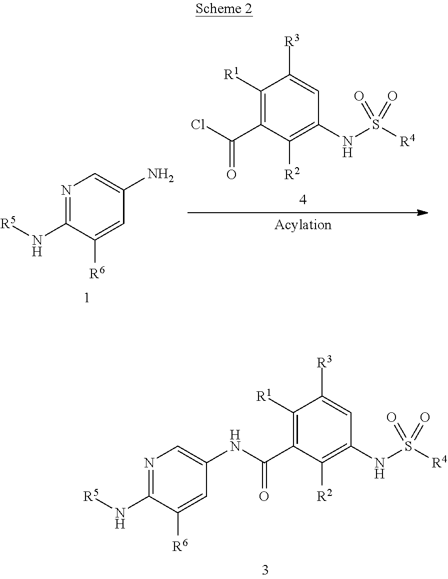

Scheme 2 shows a different method for the preparation of 6-aminopyridin-3-ylbenzamides 3, wherein R¹, R², R³, R⁴, R⁵ and R⁶ are as defined herein. Substituted 6-aminopyridine 1 may be coupled with benzoyl chloride 4 in the presence of an optional base, such as triethylamine (“TEA”), diisopropylethylamine, or pyridine, in an appropriate solvent, such as dichloromethane or tetrahydrofuran (“THF”). Benzoyl chloride 4 can be obtained by treating benzoic acid 2 (see Scheme 5) with reagents, such as thionyl chloride or oxalyl chloride, in an optional solvent, such as dichloromethane, chloroform, or toluene.

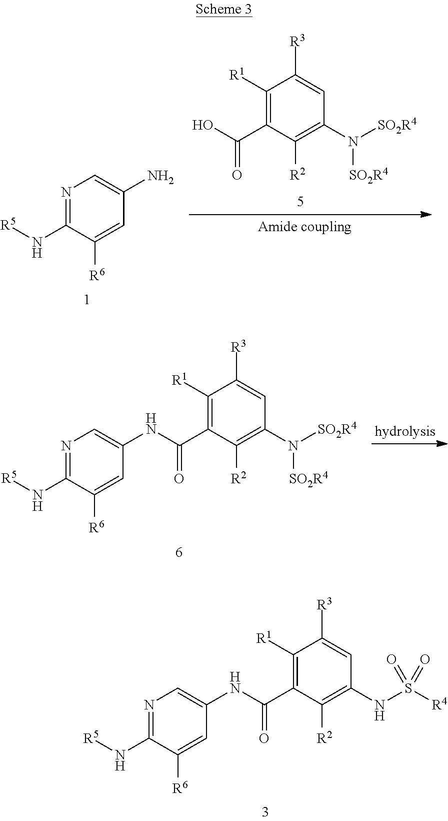

Scheme 3 illustrates another method for preparing benzamide 3, wherein R¹, R², R³, R⁴, R⁵ and R⁶ are as defined herein. Substituted 6-aminopyridine 1 may be coupled with bis-sulfonylated benzoic acid 5 using standard amide coupling conditions, such as those described in Scheme 1, to provide compound 6. Hydrolysis with a suitable base, such as aqueous sodium hydroxide or sodium carbonate, provides compound 3.

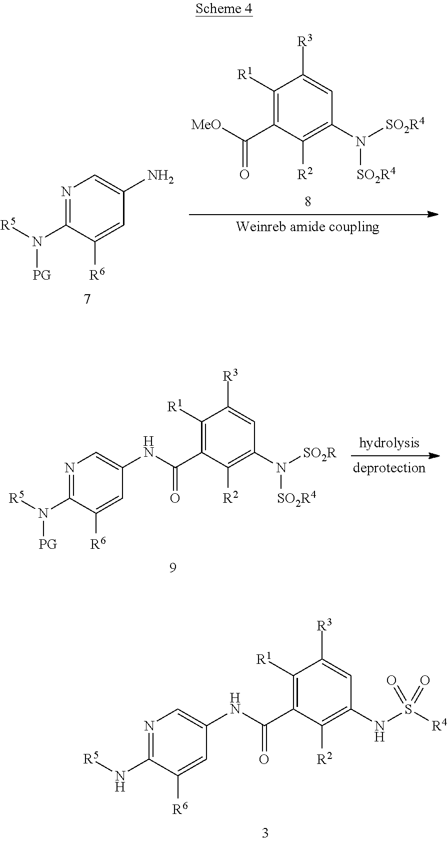

Scheme 4 illustrates yet another method for preparing benzamide 3, wherein R¹, R², R³, R⁴, R⁵ and R⁶ are as defined herein. A suitably protected substituted 6-aminopyridine 7, wherein PG is an amine protecting group, may be coupled with bis-sulfonylated benzoate 8 using Weinreb conditions (trimethylaluminum in toluene) to provide compound 9. Hydrolysis with a suitable base, such as aqueous sodium hydroxide, potassium carbonate, or sodium carbonate, and deprotection provides compound 3.

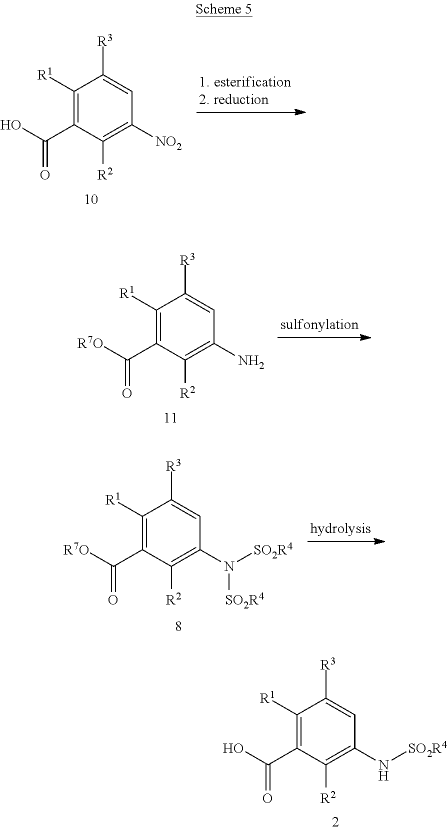

Scheme 5 shows a general method for preparing benzoate 8 and benzoic acid 2, wherein R⁷ is C₁-C₃ alkyl and R¹, R², R³ and R⁴ are as defined herein. Benzoic acid 10 is esterified by standard methods, such as by Fischer esterification conditions. The nitro group may be reduced by hydrogenation with a suitable catalyst, such as palladium on carbon. Aniline 11 may be sulfonylated with a substituted sulfonyl chloride in the presence of a suitable base, such as triethylamine, to provide benzoate 8. Hydrolysis of benzoate 8 with a base, such as aqueous sodium hydroxide, in an optional solvent, such as an alcohol (e.g., methanol), tetrahydrofuran or a mixture thereof, provides benzoic acid 2.

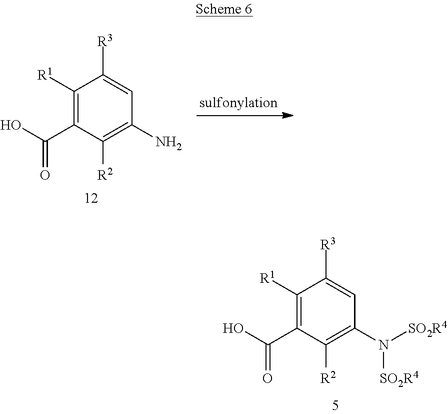

Scheme 6 shows a general method for preparing benzoic acid 5, wherein R¹, R², R³ and R⁴ are as defined herein. Aniline 12 is sulfonylated with a sulfonyl chloride in an organic solvent, such as dichloromethane, in the presence of a base, such as triethylamine, to provide compound 5.

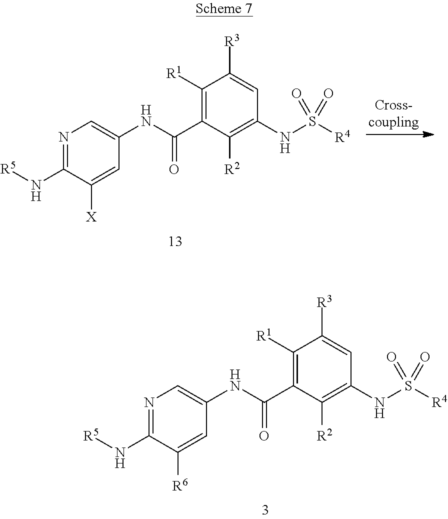

Scheme 7 illustrates a method for the installation of the R⁶ group at the end of the synthetic sequence to provide compound 3. A cross-coupling reaction with compound 13, wherein X is a halogen or triflate moiety, for example the Suzuki, Stille or Negishi reactions, in the presence of a catalyst, such as tetrakis(triphenylphosphine)palladium, can be used to install a variety of aryl and heteroaryl groups in the R⁶ position of compound 3. Compound 13 can be prepared by methods outlined in schemes 1-4, wherein R⁶ is a halogen or triflate.

Accordingly, another embodiment of the present invention provides a process for preparing compounds of Formula I, comprising:

(a) coupling a compound of Formula 1:

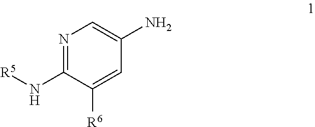

wherein R⁵ and R⁶ are as defined herein;

with a compound of Formula 2:

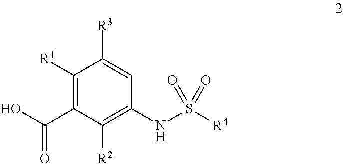

wherein R¹, R², R³ and R⁴ are as defined herein;

to provide a compound of Formula I;

(b) coupling a compound of Formula 1:

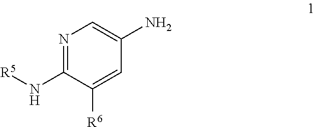

wherein R⁵ and R⁶ are as defined herein;

with a compound of Formula 4:

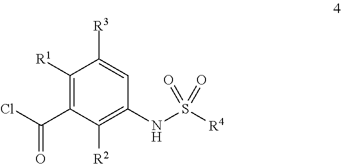

wherein R¹, R², R³ and R⁴ are as defined herein;

to provide a compound of Formula I;

(c) coupling a compound of Formula 1:

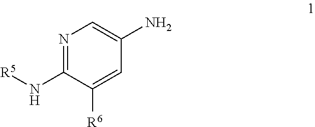

wherein R⁵ and R⁶ are as defined herein;

with a compound of Formula III:

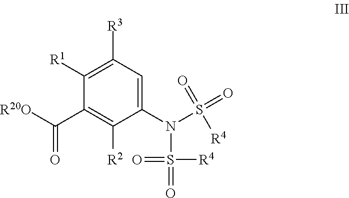

wherein R²⁰ is hydrogen, C₁-C₆ alkyl, benzyl or phenyl and R¹, R², R³ and R⁴ are as defined herein;

to provide a compound of Formula 6:

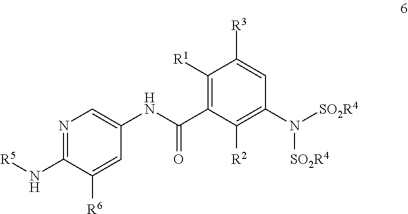

followed by hydrolysis to provide a compound of Formula I; or

(d) coupling a compound of Formula 7:

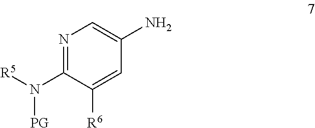

wherein PG is an amine protecting group and R⁵ and R⁶ are as defined herein;

with a compound of Formula III:

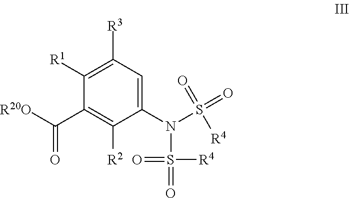

wherein R²⁰ is hydrogen, C₁-C₆ alkyl, benzyl or phenyl and R¹, R², R³ and R⁴ are as defined herein;

to provide a compound of Formula 9:

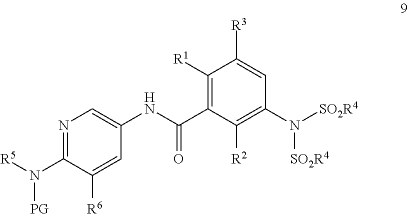

followed by hydrolysis and deprotection to provide a compound of Formula I.

In preparing compounds of Formula I, protection of remote functionalities (e.g., primary or secondary amines, etc.) of intermediates may be necessary. The need for such protection will vary depending on the nature of the remote functionality and the conditions of the preparation methods. Suitable amino-protecting groups (NH-Pg) include acetyl, trifluoroacetyl, t-butyloxycarbonyl (“Boc”), benzyloxycarbonyl (“CBz”) and 9-fluorenylmethyleneoxycarbonyl (“Fmoc”). The need for such protection is readily determined by one skilled in the art. For a general description of protecting groups and their use, see T. W. Greene, et al. Greene's Protective Groups in Organic Synthesis. New York: Wiley Interscience, 2006.

### Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (Eliel, E. and Wilen, S. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., Ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). Enriched or purified enantiomers can be distinguished by methods used to distinguish other chiral molecules with asymmetric carbon atoms, such as optical rotation and circular dichroism.

### Biological Evaluation

B-Raf mutant protein 447-717 (V600E) was co-expressed with the chaperone protein Cdc37, complexed with Hsp90 (Roe, S. Mark, et al. “The Mechanism of Hsp90 Regulation by the Protein Kinase-Specific Cochaperone p50^{cdc37}.” Cell. Vol. 116 (2004): pp. 87-98; Stancato, L F, et al. “Raf exists in a native heterocomplex with Hsp90 and p50 that can be reconstituted in a cell free system.” J. Biol. Chem. 268(29) (1993): pp. 21711-21716).

Determining the activity of Raf in the sample is possible by a number of direct and indirect detection methods (US 2004/0082014). Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. The activity/inhibition of V600E full-length B-Raf was estimated by measuring the incorporation of radio labeled phosphate from [γ-³³P]ATP into FSBA-modified wild-type MEK (see Example A).

### Administration and Pharmaceutical Formulations

The compounds of the invention may be administered by any convenient route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal.

The compounds may be administered in any convenient administrative form, e.g. tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents. If parenteral administration is desired, the compositions will be sterile and in a solution or suspension form suitable for injection or infusion.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

One embodiment of the present invention includes a pharmaceutical composition comprising a compound of Formula I, or a stereoisomer or pharmaceutically acceptable salt thereof. In a further embodiment, the present invention provides a pharmaceutical composition comprising a compound of Formula I, or a stereoisomer or pharmaceutically acceptable salt thereof, together with a pharmaceutically acceptable carrier or excipient.

Another embodiment of the present invention provides a pharmaceutical composition comprising a compound of Formula I for use in the treatment of a hyperproliferative disease.

Another embodiment of the present invention provides a pharmaceutical composition comprising a compound of Formula I for use in the treatment of cancer.

Methods of Treatment with Compounds of the Invention

The invention includes methods of treating or preventing disease or condition by administering one or more compounds of this invention, or a stereoisomer or pharmaceutically acceptable salt thereof. In one embodiment, a human patient is treated with a compound of Formula I, or a stereoisomer or pharmaceutically acceptable salt thereof, and a pharmaceutically acceptable carrier, adjuvant, or vehicle in an amount to detectably inhibit B-Raf activity.

In another embodiment, a human patient is treated with a compound of Formula I, or a stereoisomer, tautomer or pharmaceutically acceptable salt thereof, and a pharmaceutically acceptable carrier, adjuvant, or vehicle in an amount to detectably inhibit B-Raf activity.

In another embodiment of the present invention, a method of treating a hyperproliferative disease in a mammal comprising administering a therapeutically effective amount of the compound of Formula I, or a stereoisomer, tautomer or pharmaceutically acceptable salt thereof, to the mammal is provided.

In another embodiment of the present invention, a method of treating a hyperproliferative disease in a mammal comprising administering a therapeutically effective amount of the compound of Formula I, or a stereoisomer or pharmaceutically acceptable salt thereof, to the mammal is provided.

In another embodiment of the present invention, a method of treating kidney disease in a mammal comprising administering a therapeutically effective amount of the compound of Formula I, or a stereoisomer, tautomer or pharmaceutically acceptable salt thereof, to the mammal is provided. In a further embodiment, the kidney disease is polycystic kidney disease.

In another embodiment, a method of treating or preventing cancer in a mammal in need of such treatment, wherein the method comprises administering to said mammal a therapeutically effective amount of a compound of Formula I, or a stereoisomer or pharmaceutically acceptable salt thereof. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. Another embodiment of the present invention provides the use of a compound of Formula I, or a stereoisomer or pharmaceutically acceptable salt thereof, in the manufacture of a medicament for the treatment of cancer.

In another embodiment, a method of treating or preventing cancer in a mammal in need of such treatment, wherein the method comprises administering to said mammal a therapeutically effective amount of a compound of Formula I, or a stereoisomer, tautomer or pharmaceutically acceptable salt thereof.

Another embodiment of the present invention provides the use of a compound of Formula I, or a stereoisomer, tautomer or pharmaceutically acceptable salt thereof, in the manufacture of a medicament for the treatment of cancer.

Another embodiment of the present invention provides the use of a compound of Formula I, or a stereoisomer, tautomer or pharmaceutically acceptable salt thereof, in the manufacture of a medicament for the treatment of kidney disease. In a further embodiment, the kidney disease is polycystic kidney disease.

In another embodiment, a method of preventing or treating cancer, comprising administering to a mammal in need of such treatment an effective amount of a compound of Formula I, or a stereoisomer, tautomer or pharmaceutically acceptable salt thereof, alone or in combination with one or more additional compounds having anti-cancer properties.

In another embodiment, a method of preventing or treating cancer, comprising administering to a mammal in need of such treatment an effective amount of a compound of Formula I, or a stereoisomer or pharmaceutically acceptable salt thereof, alone or in combination with one or more additional compounds having anti-cancer properties.

In one further embodiment, the cancer is a sarcoma.

In another further embodiment, the cancer is a carcinoma. In one further embodiment, the carcinoma is squamous cell carcinoma. In another further embodiment, the carcinoma is an adenoma or adenocarcinoma.

In another embodiment, a method of treating or preventing a disease or disorder modulated by B-Raf, comprising administering to a mammal in need of such treatment an effective amount of a compound of Formula I, or a stereoisomer or pharmaceutically acceptable salt thereof. Examples of such diseases and disorders include, but are not limited to, cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

In another embodiment, a method of treating or preventing a disease or disorder modulated by B-Raf, comprising administering to a mammal in need of such treatment an effective amount of a compound of Formula I, or a stereoisomer, tautomer or pharmaceutically acceptable salt thereof.

In another embodiment of the present invention, a method of preventing or treating kidney disease, comprising administering to a mammal in need of such treatment an effective amount of Formula I, or a stereoisomer, tautomer or pharmaceutically acceptable salt thereof, alone or in combination with one or more additional compounds. In another embodiment of the present invention, a method of preventing or treating polycystic kidney disease, comprising administering to a mammal in need of such treatment an effective amount of a compound of Formula I, or a stereoisomer, tautomer or pharmaceutically acceptable salt thereof, alone or in combination with one or more additional compounds.

Another embodiment of the present invention provides the use of a compound of Formula I, or a stereoisomer or pharmaceutically acceptable salt thereof, in the manufacture of a medicament for the treatment of cancer. The cancer is selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia. In a further embodiment, the use of a compound of Formula I in the manufacture of a medicament, for use as a b-Raf inhibitor in the treatment of a patient undergoing cancer therapy.

Another embodiment of the present invention provides the use of a compound of Formula I, or a stereoisomer, tautomer or pharmaceutically acceptable salt thereof, in the manufacture of a medicament for the treatment of cancer.

Another embodiment of the present invention provides the use of a compound of Formula I, or a stereoisomer, tautomer or pharmaceutically acceptable salt thereof, in the manufacture of a medicament for the treatment of polycystic kidney disease. In a further embodiment, the kidney disease is polycystic kidney disease.

Another embodiment of the present invention provides the compounds of Formula I for use in therapy.

Another embodiment of the present invention provides the compounds of Formula I for use in the treatment of a hyperproliferative disease. In a further embodiment, the hyperproliferative disease is cancer (as further defined and may be individually selected from those above).

Another embodiment of the present invention provides the compounds of Formula I for use in the treatment of kidney disease. In a further embodiment, the kidney disease is polycystic kidney disease.

### Combination Therapy

The compounds of this invention and stereoisomers and pharmaceutically acceptable salts thereof may be employed alone or in combination with other therapeutic agents for treatment. The compounds of the present invention can be used in combination with one or more additional drugs, for example an anti-hyperproliferative, anti-cancer or chemotherapeutic agent. The second compound of the pharmaceutical combination formulation or dosing regimen preferably has complementary activities to the compound of this invention such that they do not adversely affect each other. Such agents are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

A “chemotherapeutic agent” is a chemical compound useful in the treatment of cancer, regardless of mechanism of action. Chemotherapeutic agents include compounds used in “targeted therapy” and conventional chemotherapy. A number of suitable chemotherapeutic agents to be used as combination therapeutics are contemplated for use in the methods of the present invention. The present invention contemplates, but is not limited to, administration of numerous anticancer agents, such as: agents that induce apoptosis; polynucleotides (e.g., ribozymes); polypeptides (e.g., enzymes); drugs; biological mimetics; alkaloids; alkylating agents; antitumor antibiotics; antimetabolites; hormones; platinum compounds; monoclonal antibodies conjugated with anticancer drugs, toxins, and/or radionuclides; biological response modifiers (e.g., interferons [e.g., IFN-a, etc.] and interleukins [e.g., IL-2, etc.], etc.); adoptive immunotherapy agents; hematopoietic growth factors; agents that induce tumor cell differentiation (e.g., all-trans-retinoic acid, etc.); gene therapy reagents; antisense therapy reagents and nucleotides; tumor vaccines; inhibitors of angiogenesis, and the like.

Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sunitinib (SUTENT®, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (NEXAVAR®, Bayer), Irinotecan (CAMPTOSAR®), Pfizer) and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE™ (Cremophor-free), albumin-engineered nanoparticle formulations of paclitaxel (American Pharmaceutical Partners, Schaumberg, Ill.), and TAXOTERE® (doxetaxel; Rhône-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the Raf inhibitors of the invention include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

### EXAMPLES

In order to illustrate the invention, the following Examples are included. However, it is to be understood that these Examples do not limit the invention and are only meant to suggest a method of practicing the invention. Persons skilled in the art will recognize that the chemical reactions described may be readily adapted to prepare a number of other compounds of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

In the Examples described below, unless otherwise indicated all temperatures are set forth in degrees Celsius. Reagents were purchased from commercial suppliers such as Sigma-Aldrich, Alfa Aesar, or TCI, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography purification was done on a Biotage system (Manufacturer: Dyax Corporation) having a silica gel column or on a silica SepPak cartridge (Waters) or on a Teledyne Isco Combiflash purification system using prepacked silica gel cartridges. ¹H NMR spectra were recorded on a Varian instrument operating at 400 MHz. ¹H-NMR spectra were obtained as CDCl₃, CD₂Cl₂, CD₃OD, D₂O, d₆-DMSO or d₆-acetone solutions (reported in ppm), using tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05 ppm) as the reference standard. When peak multiplicities are reported, the following abbreviations are used: s (singlet), d (doublet), t (triplet), q (quartet), sx (sextuplet), qn (quintuplet), sx (sextuplet), m (multiplet), br (broadened), dd (doublet of doublets), dt (doublet of triplets). Coupling constants, when given, are reported in Hertz (Hz).

### Example A

### B-Raf IC₅₀ Assay Protocol

Activity of human recombinant B-Raf protein may be assessed in vitro by assay of the incorporation of radio labeled phosphate to recombinant MAP kinase (MEK), a known physiologic substrate of B-Raf, according to US 2004/0127496 and WO 03/022840. Catalytically active human recombinant B-Raf protein is obtained by purification from sf9 insect cells infected with a human B-Raf recombinant baculovirus expression vector.

The activity/inhibition of V600E full-length B-Raf was estimated by measuring the incorporation of radio labeled phosphate from [γ-³³P]ATP into FSBA-modified wild-type MEK. The 30-μL assay mixtures contained 25 mM Na Pipes, pH 7.2, 100 mM KCl, 10 mM MgCl₂, 5 mM β-glycerophosphate, 100 μM Na Vanadate, 4 μM ATP, 500 nCi [γ-³³P]ATP, 1 μM FSBA-MEK and 20 nM V600E full-length B-Raf. Incubations were carried out at 22° C. in a Costar 3365 plate (Corning). Prior to the assay, the B-Raf and FSBA-MEK were preincubated together in assay buffer at 1.5×(20 μL, of 30 nM and 1.5 μM, respectively) for 15 minutes, and the assay was initiated by the addition of 10 μL of 10 μM ATP. Following the 60-minute incubation, the assay mixtures were quenched by the addition of 100 μl, of 25% TCA, the plate was mixed on a rotary shaker for 1 minute, and the product was captured on a Perkin-Elmer GF/B filter plate using a Tomtec Mach III Harvester. After sealing the bottom of the plate, 35 μL of Bio-Safe II (Research Products International) scintillation cocktail were added to each well and the plate was top-sealed and counted in a Topcount NXT (Packard).

The compounds of Examples 1-12 were tested in the above assay and found to have an IC₅₀ of less than 1 μM.

### Example B

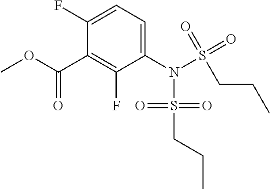

### methyl 2,6-difluoro-3-(N-(propylsulfonyl)propylsulfonamido)benzoate

Step A: A 1 L flask was charged with 2,6-difluoro-3-nitrobenzoic acid (17.0 g, 83.7 mmol) and MeOH (170 mL, 0.5M). The flask was placed in a cold water bath, and an addition funnel charged with a 2M solution of trimethylsilyl (“TMS”) diazomethane in hexanes (209 mL, 419 mmol) was attached to the flask. The TMS diazomethane solution was added slowly to the reaction flask over the course of 2 hours. A large excess of reagent was required in order for the reaction to reach completion as determined by the ceased evolution of N₂ upon further addition of reagent. The volatiles were removed in vacuo to afford methyl 2,6-difluoro-3-nitrobenzoate as a solid (18.2 g, 99%). The material was taken directly onto Step B.

Step B: 10% (wt) Pd on activated carbon (4.46 g, 4.19 mmol) was added to a 1 L flask charged with methyl 2,6-difluoro-3-nitrobenzoate (18.2 g, 83.8 mmol) under a nitrogen atmosphere. EtOH (350 mL, 0.25M) was added, and then H₂ was passed through the reaction mixture for 15 minutes. The reaction mixture was stirred under two H₂ balloons overnight. The following day the reaction mixture was re-flushed with fresh H₂ balloons and stirred an additional 4 hours. Upon consumption of the starting material and intermediate hydroxylamine as determined by thin layer chromatography (“TLC”), N₂ gas was flushed through the reaction mixture. The mixture was then filtered through glass microfibre filter (“GF/F”) paper twice. The volatiles were removed to afford methyl 3-amino-2,6-difluorobenzoate as an oil (15.66 g, 99%). The material was taken directly onto the next step.

Step C: propane-1-sulfonyl chloride (23.46 mL, 209.3 mmol) was slowly added to a solution of methyl 3-amino-2,6-difluorobenzoate (15.66 g, 83.7 mmol) and triethylamine (35.00 mL, 251.1 mmol) in CH₂Cl₂ (175 mL, 0.5M) maintained in a cool water bath. The reaction mixture was stirred for 1 hour at room temperature. Water (300 mL) was added and the organic layer was separated, washed with water (2×300 mL) and brine (200 mL), then dried (Na₂SO₄), filtered and concentrated to an oil. The crude product was purified by column chromatography, eluting with 15% ethyl acetate/hexanes. The isolated fractions were triturated with hexanes to afford methyl 2,6-difluoro-3-(N-(propylsulfonyl)propylsulfonamido)-benzoate as a solid (24.4 g, 73% yield for 3 steps). ¹H NMR (400 MHz, CDCl₃) δ 7.52-7.45 (m, 1H), 7.08-7.02 (m, 1H), 3.97 (s, 3H), 3.68-3.59 (m, 2H), 3.53-3.45 (m, 2H), 2.02-1.89 (m, 4H), 1.10 (t, J=7.4 Hz, 6H). m/z (APCI-neg) M-(SO₂Pr)=292.2.

### Example C

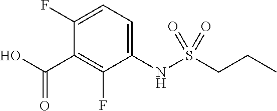

### 2,6-difluoro-3-(propylsulfonamido)benzoic acid

A 1N aqueous NaOH solution (150 mL, 150 mmol) was added to a solution of methyl 2,6-difluoro-3-(N-(propylsulfonyl)propylsulfonamido)-benzoate (20.0 g, 50.1 mmol) in 4:1 THF/MeOH (250 mL, 0.2M). The reaction mixture was stirred at room temperature overnight. The majority of the organic solvents were then removed in vacuo (water bath temperature 35° C.). 1N HCl (150 mL) was slowly added to the mixture, and the resulting solid was filtered and rinsed with water (4×50 mL). The material was then washed with Et₂O (4×15 mL) to give 2,6-difluoro-3-(propylsulfonamido)benzoic acid as a solid (10.7 g, 77% yield). ¹H NMR (400 MHz, d₆-DMSO) δ 9.74 (s, 1H), 7.57-7.50 (m, 1H), 7.23-7.17 (m, 1H), 3.11-3.06 (m, 2H), 1.79-1.69 (m, 2H), 0.98 (t, J=7.4 Hz, 3H). m/z (APC)-neg) M−1=278.0.

### Example D

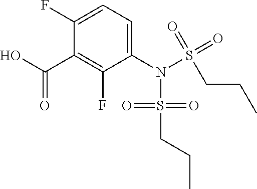

### 2,6-difluoro-3-(N-(propylsulfonyl)propylsulfonamido)benzoic acid

Propane-1-sulfonyl chloride (1.225 mL, 10.92 mmol) was added to a mixture of 3-amino-2,6-difluorobenzoic acid (0.573 g, 3.310 mmol), TEA (2.030 mL, 14.56 mmol) and CH₂Cl₂ (17 mL, 0.2M) cooled to 0° C. The reaction mixture was allowed to warm to room temperature and stirred for 1 hour. The mixture was then partitioned between saturated NaHCO₃ (100 mL) and ethyl acetate (75 mL). The aqueous layer was washed with ethyl acetate (50 mL) and then acidified with concentrated HCl to a pH of about 1. The acidified aqueous layer was extracted with ethyl acetate (2×50 mL), and the combined ethyl acetate extracts were dried (Na₂SO₄), filtered and concentrated. The resulting residue was triturated with hexanes to afford 2,6-difluoro-3-(N-(propylsulfonyl)propylsulfonamido)benzoic acid as a solid (0.948 g, 74% yield). ¹H NMR (400 MHz, d₆-DMSO) δ 7.90-7.84 (m, 1H), 7.39-7.34 (m, 1H), 3.73-3.58 (m, 4H), 1.88-1.74 (m, 4H), 1.01 (t, J=7.5 Hz, 6H). m/z (APC)-neg) M-(SO₂Pr)=278.1.

### Example E

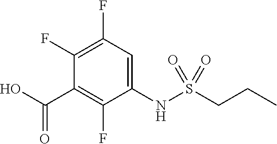

### 2,3,6-trifluoro-5-(propylsulfonamido)benzoic acid

2,3,6-Trifluoro-5-(propylsulfonamido)benzoic acid (8.5%) was prepared according to the general procedure of Example D, substituting 3-amino-2,5,6-trifluorobenzoic acid for 3-amino-2,6-difluorobenzoic acid.

### Example F

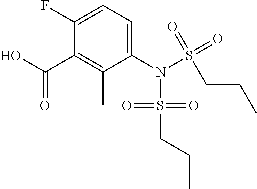

### 6-fluoro-2-methyl-3-(N-(propylsulfonyl)propylsulfonamido)benzoic acid

6-Fluoro-2-methyl-3-(N-(propylsulfonyl)propylsulfonamido)benzoic acid

(11%) was prepared according to the general procedure of Example D, substituting 3-amino-6-fluoro-2-methylbenzoic acid for 3-amino-2,6-difluorobenzoic acid.

### Example G

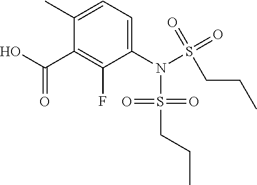

### 2-fluoro-6-methyl-3-(N-(propylsulfonyl)propylsulfonamido)benzoic acid

2-Fluoro-6-methyl-3-(N-(propylsulfonyepropylsulfonamido)benzoic acid (3%) was prepared according to the general procedure of Example D, substituting 3-amino-2-fluoro-6-methylbenzoic acid for 3-amino-2,6-difluorobenzoic acid.

### Example H

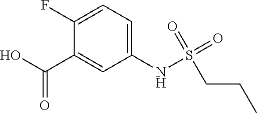

### 2-fluoro-5-(propylsulfonamido)benzoic acid

Propane-1-sulfonyl chloride (0.0871 mL, 0.774 mmol) was dissolved in 10% Na₂CO₃ (1.65 mL, 1.55 mmol) at room temperature. 5-Amino-2-fluorobenzoic acid (0.100 g, 0.645 mmol) was added and heated to 60° C. overnight. Propane-1-sulfonyl chloride (0.0871 mL, 0.774 mmol) was added again, and the reaction mixture was heated at 60° C. for another hour. The reaction mixture was cooled to room temperature, diluted with water, taken to a pH of 10 with 10% Na₂CO₃ and extracted with DCM (2×). The reaction mixture was then taken to a pH of 2 with 1N HCl, extracted with DCM (3×) and concentrated to a solid, 2-fluoro-5-(propylsulfonamido)benzoic acid (29%).

### Example I

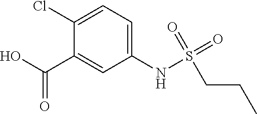

### 2-chloro-5-(propylsulfonamido)benzoic acid

2-Chloro-5-(propylsulfonamido)benzoic acid (14%) was prepared according to the general procedure for Example H, substituting 5-amino-2-chlorobenzoic acid for 5-amino-2-fluorobenzoic acid.

### Example J

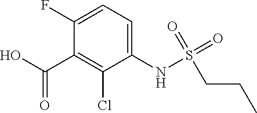

### 2-chloro-6-fluoro-3-(propylsulfonamido)benzoic acid

Step A: 2-Chloro-6-fluorobenzoic acid (2.00 g, 11.5 mmol) was dissolved in sulfuric acid (20 mL) and cooled to 0° C. Nitric acid (0.529 mL, 12.6 mmol) was added, and the reaction mixture was warmed to room temperature for one hour. The reaction mixture was diluted with water, and the aqueous portion was extracted with DCM (3×), dried over Na₂SO₄, concentrated to a solid, 2-chloro-6-fluoro-3-nitrobenzoic acid (97%), which was used directly in the next step without further purification.

Step B: 2-Chloro-6-fluoro-3-nitrobenzoic acid (0.100 g, 0.455 mmol) and Zn dust (0.298 g, 4.55 mmol) were taken up in THF (4 mL) and saturated aqueous NH₄Cl (2 mL) and stirred at room temperature overnight. The reaction mixture was filtered through Celite, concentrated to a solid, and dissolved in water. The pH was adjusted to 2 with 1N HCl, and the aqueous portion was extracted with DCM (3×). The organic portion was dried over Na₂SO₄ and concentrated to a solid, 3-amino-2-chloro-6-fluorobenzoic acid (49%), which was used directly in the next step without further purification.

Step C: 2-Chloro-6-fluoro-3-(propylsulfonamido)benzoic acid (13%) was prepared according to the general procedure for Example H, substituting 3-amino-2-chloro-6-fluorobenzoic acid for 5-amino-2-fluorobenzoic acid.

### Example K

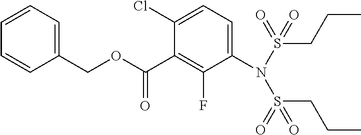

### benzyl 6-chloro-2-fluoro-3-(N-(propyl sulfonyl)propylsulfonamido)benzoate

Step A: A flame dried flask equipped with a stir bar and rubber septum was charged with 4-chloro-2-fluoroaniline (5.00 g, 34.35 mmol) and dry THF (170 mL). This solution was chilled to −78° C., and n-BuLi (14.7 mL, 1.07 eq. of 2.5M solution in hexanes) was then added over a 15 minute period. This mixture was stirred at −78° C. for 20 minutes, and then a THF solution (25 mL) of 1,2-bis(chlorodimethylsilyl)ethane (7.76 g, 1.05 eq.) was added slowly (over a 10 minute period) to the reaction mixture. This was stirred for 1 hour, and then 2.5M n-BuLi in hexanes (15.11 mL, 1.1 eq.) was added slowly. After allowing the mixture to warm to room temperature for one hour, the mixture was chilled back to −78° C. A third allotment of n-BuLi (15.66 mL, 1.14 eq.) was added slowly, and the mixture was stirred at −78° C. for 75 minutes. Benzyl chloroformate (7.40 g, 1.2 eq.) was then added slowly, and the mixture was stirred at −78° C. for one hour. The cooling bath was then removed. The mixture was allowed to warm for 30 minutes and then quenched with water (70 mL) and concentrated HCl (25 mL). The mixture was allowed to continue to warm to room temperature. The mixture was then extracted with ethyl acetate (“EtOAc”). The extracts were washed twice with a saturated Na₂HCO₃ solution, once with water, dried over sodium sulfate and concentrated. The resulting residue was flashed on a 65 Biotage (30% ethyl acetate/hexane) to produce benzyl 3-amino-6-chloro-2-fluorobenzoate (4.3 g, 45%) as an oil. ¹H NMR (DMSO-d₆, 400 MHz) δ 7.37-7.48 (m, 5H), 7.07 (dd, 1H, J=8, 2), 6.87 (t, 1H, J=8), 5.61 (br s, 2H), 5.40 (s, 2H).

Step B: Benzyl 3-amino-6-chloro-2-fluorobenzoate (4.3 g, 15.37 mmol) was dissolved in dry dichloromethane (270 mL). Triethylamine (5.36 mL, 2.5 eq.) was added, and the mixture was chilled to 0° C. Propane-1-sulfonyl chloride (3.63 mL, 32.3 mmol, 2.1 eq.) was then added via syringe, and a precipitate resulted. Once the addition was complete, the mixture was allowed to warm to room temperature, and the starting material was consumed as determined by TLC (3:1 hexane:ethyl acetate). The mixture was then diluted with dichloromethane (200 mL), washed with 2M aqueous HCl (2×100 mL), saturated Na₂HCO₃ solution, dried over sodium sulfate and concentrated. The resulting residue was purified on a 65 Biotage chromatography system (40% ethyl acetate/hexane) to produce benzyl 6-chloro-2-fluoro-3-(N-(propylsulfonyl)propylsulfonamido)benzoate (5.5 g, 72%) as an oil that slowly solidified upon standing. NMR (CDCl₃, 400 MHz) δ 7.28-7.45 (m, 7H), 5.42 (s, 2H), 3.58-3.66 (m, 2H), 3.43-3.52 (m, 2H), 1.08 (t, 6H, J=8).

### Example L

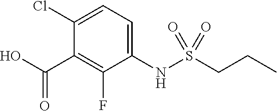

### 6-chloro-2-fluoro-3-(propylsulfonamido)benzoic acid

Benzyl 6-chloro-2-fluoro-3-(N-(propylsulfonyl)propylsulfonamido)benzoate (5.4 g, 10.98 mmol) was dissolved in THF (100 mL) and 1M aqueous KOH (100 mL). This mixture was refluxed for 16 hours and then allowed to cool to room temperature. The mixture was then acidified to a pH of 2 with 2M aqueous HCl and extracted with EtOAc (2 X). The extracts were washed with water, dried over sodium sulfate and concentrated to a solid that was triturated with hexanes/ether to give 6-chloro-2-fluoro-3-(propylsulfonamido)benzoic acid (2.2 g, 68%) as a solid. NMR (DMSO-d₆, 400 MHz) δ 9.93 (s, 1H), 7.49 (t, 1H, J=8), 7.38 (dd, 1H, J=8, 2), 3.11-3.16 (m, 2H), 1.68-1.78 (m, 2H), 0.97 (t, 3H, J=8).

### Example M

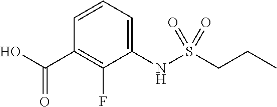

### 2-fluoro-3-(propylsulfonamido)benzoic acid

6-Chloro-2-fluoro-3-(propylsulfonamido)benzoic acid (0.5 g, 1.69 mmol) was dissolved in methanol (15 mL), and Pearlman's catalyst (one weight equivalent, 0.5 g, 20% Pd(OH)₂ on carbon, 50% by weight water) was added. This mixture was subjected to a balloon of hydrogen for 3 hours and then filtered through GF/F filter paper. The filtrate was concentrated to 2-fluoro-3-(propylsulfonamido)benzoic acid (396 mg, 90%) as a solid. MS (M-H⁺) 262. NMR (DMSO-d₆, 400 MHz) δ 13.36 (s, 1H), 9.76 (s, 1H), 7.58-7.70 (m, 2H), 7.26 (t, 1H, J=8), 3.10 (t, 2H, J=8), 1.69-1.80 (m, 2H), 0.98 (t, 3H, J=8).

### Example N

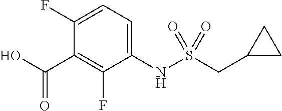

### 3-(cyclopropylmethylsulfonamido)-2,6-difluorobenzoic acid

Step A: Cyclopropylmethanesulfonyl chloride (1.27 g, 8.20 mmol) was added to a mixture of 3-amino-2,6-difluorobenzoic acid (0.430 g, 2.48 mmol), triethylamine (1.52 mL, 10.9 mmol) and CH₂Cl₂ (12 mL, 0.2M) cooled to 0° C. The reaction mixture was allowed to warm to room temperature and stirred for 1 hour. The mixture was then partitioned between saturated NaHCO₃ (75 mL) and ethyl acetate (50 mL). The aqueous layer was washed with ethyl acetate (50 mL) and then acidified to a pH of 1 with concentrated HCl. The acidified aqueous layer was extracted twice with ethyl acetate (2×50 mL), and the combined ethyl acetate extracts were dried (Na₂SO₄), filtered and concentrated to provide crude 3-(1-cyclopropyl-N-(cyclopropylmethylsulfonyl)methylsulfonamido)-2,6-difluorobenzoic acid (380 mg, 37%).

Step B: A solution of 1N NaOH (2.78 mL, 2.78 mmol) was added to a solution of crude 3-(1-cyclopropyl-N-(cyclopropylmethylsulfonyl)methylsulfonamido)-2,6-difluorobenzoic acid (380 mg, 0.928 mmol) in 4:1 THF/MeOH (5 mL, 0.2M). The reaction mixture was stirred at room temperature for 1 hour, after which most of the organic solvents were removed. 1N HCl (3 mL) was slowly added to the mixture to acidify to a pH of 1. The acidified aqueous layer was extracted with ethyl acetate (75 mL). The ethyl acetate extract was washed with water (2×20 mL), brine (20 mL), dried (Na₂SO₄), filtered and concentrated. Trituration of the residue with Et₂O afforded 3-(cyclopropylmethylsulfonamido)-2,6-difluorobenzoic acid as a solid (139 mg, 51%). ¹H NMR (400 MHz, d₆-DMSO) δ 9.76 (s, 1H), 7.60-7.54 (m, 1H), 7.22-7.16 (m, 1H), 3.10 (d, J=7.0 Hz, 2H), 1.10-0.99 (m, 1H), 0.58-0.53 (m, 2H), 0.36-0.31 (m, 2H); m/z (APC)-neg) M−1=289.9.

### Example O

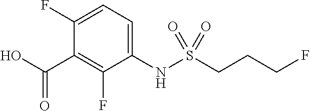

### 2,6-difluoro-3-(3-fluoropropylsulfonamido)benzoic acid

Methyl 2,6-difluoro-3-(N-(3-fluoropropylsulfonyl)-3-fluoropropylsulfonamido)benzoate was made according to the general procedure for Example B, substituting 3-fluoropropyl sulfonyl chloride for propane-1-sulfonyl chloride. ¹H NMR (400 MHz, DMSO-d₆) δ 8.05-7.99 (m, 1H), 7.44 (t, 1H), 4.62 (t, 2H), 4.50 (t, 2H), 3.93 (s, 3H), 3.89-3.74 (m, 4H), 2.26-2.11 (m, 4H).

2,6-Difluoro-3-(3-fluoropropylsulfonamido)benzoic acid was prepared according to the general procedure for Example C, substituting methyl 2,6-difluoro-3-(N-(3-fluoropropylsulfonyl)-3-fluoropropylsulfonamido)benzoate for methyl 2,6-difluoro-3-(N-(propylsulfonyl)-propylsulfonamido)benzoate. ¹H NMR (500 MHz, DMSO-d₆) δ 14.05 (br s, 1H), 9.71 (s, 1H), 7.56-7.50 (m, 1H), 7.20 (t, 1H), 3.12-3.08 (m, 2H), 1.73-1.66 (m, 2H), 1.39 (sx, 2H), 0.87 (t, 3H).

### Example P

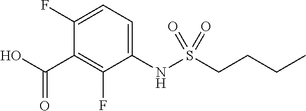

### 3-(butylsulfonamido)-2,6-difluorobenzoic acid

Methyl 2,6-difluoro-3-(N-(butylsulfonyl)-butylsulfonamido)benzoate was made according to the general procedure for Example B, substituting butane-1-sulfonyl chloride for propane-1-sulfonyl chloride. ¹H NMR (500 MHz, DMSO-d₆) δ 7.99-7.94 (m, 1H), 7.42 (t, 1H), 3.92 (s, 3H), 3.74-3.62 (m, 4H), 1.81-1.68 (m, 4H), 1.42 (sx, 4H), 0.89 (t, 6H).

3-(Butylsulfonamido)-2,6-difluorobenzoic acid was prepared according to the general procedure for Example C, substituting methyl 2,6-difluoro-3-(N-(butylsulfonyl)-butylsulfonamido)benzoate for methyl 2,6-difluoro-3-(N-(propylsulfonyl)-propylsulfonamido)benzoate. ¹H NMR (400 MHz, DMSO-d₆) δ 14.05 (br s, 1H), 9.71 (s, 1H), 7.56-7.50 (m, 1H), 7.20 (t, 1H), 3.12-3.08 (m, 2H), 1.73-1.66 (m, 2H), 1.39 (sx, 2H), 0.87 (t, 3H).

### Example Q

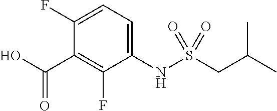

### 2,6-difluoro-3-(2-methylpropylsulfonamido)benzoic acid

Methyl-2,6-difluoro-3-(N-(2-methylpropylsulfonyl)-2-methylpropyl-sulfonamido)benzoate was made according to the general procedure for Example B, substituting 2-methylpropyl sulfonyl chloride for propane-1-sulfonyl chloride. m/z (LC-MS) M+1=428.4.

2,6-Difluoro-3-(2-methylpropylsulfonamido)benzoic acid was prepared according to the general procedure for Example C, substituting methyl-2,6-difluoro-3-(N-(2-methylpropylsulfonyl)-2-methylpropylsulfonamido)benzoate for methyl 2,6-difluoro-3-(N-(propylsulfonyl)-propylsulfonamido)benzoate. ¹H NMR (400 MHz, DMSO-d₆) δ 14.01 (s, 1H), 9.71 (s, 1H), 7.56 (dd, 1H), 7.22 (dd, 1H), 3.02 (d, 2H), 2.18-2.15 (m, 1H), 1.03 (d, 6H); m/z (LC-MS) M+1=294.3.

### Example R

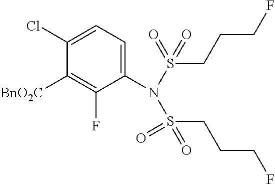

### benzyl 6-chloro-2-fluoro-3-(3-fluoro-N-(3-fluoropropyl sulfonyl)propylsulfonamido)benzoate

Benzyl 6-chloro-2-fluoro-3-(3-fluoro-N-(3-fluoropropylsulfonyl)propylsulfonamido)benzoate (92%) was prepared according to the general procedure for Example K, Step B substituting 3-fluoropropane-1-sulfonyl chloride for propane-1-sulfonyl chloride.

### Example S

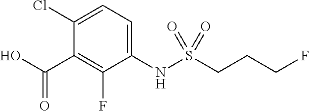

### 6-chloro-2-fluoro-3-(3-fluoropropylsulfonamido)benzoic acid

6-Chloro-2-fluoro-3-(3-fluoropropylsulfonamido)benzoic acid (71%) was prepared according to the general procedure for Example L substituting benzyl 6-chloro-2-fluoro-3-(3-fluoro-N-(3-fluoropropylsulfonyl)propylsulfonamido)benzoate for benzyl 6-chloro-2-fluoro-3-(N-(propylsulfonyl)propylsulfonamido)benzoate.

### Example T

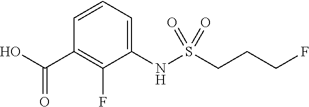

### 2-fluoro-3-(3-fluoropropylsulfonamido)benzoic acid

2-Fluoro-3-(3-fluoropropylsulfonamido)benzoic acid (81%) was prepared according to the general procedure for Example M substituting 6-chloro-2-fluoro-3-(3-fluoropropylsulfonamido)benzoic acid for 6-chloro-2-fluoro-3-(propylsulfonamido)benzoic acid.

### Example U

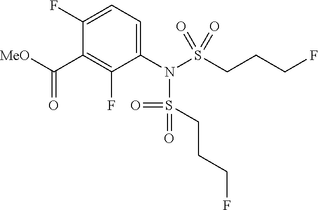

### methyl 2,6-difluoro-3-(3-fluoro-N-(3-fluoropropyl sulfonybpropylsulfonamido)benzoate

3-Fluoropropane-1-sulfonyl chloride (14.3 mL, 129 mmol) was slowly added to a solution of methyl 3-amino-2,6-difluorobenzoate (24.1 g, 129 mmol) and pyridine (31.2 mL, 386 mmol) in CH₂Cl₂ (360 mL). The reaction mixture was stirred for over two days at room temperature. The reaction mixture was diluted with methylene chloride. The reaction mixture was then washed with an aqueous solution of saturated sodium bicarbonate, 1N HCl, and brine, then dried (Na₂SO₄), filtered and concentrated to an oil to give methyl 2,6-difluoro-3-(3-fluoro-N-(3-fluoropropylsulfonyl)propylsulfonamido)benzoate (38.1 g). ¹H NMR (400 MHz, CDCl₃, ppm) 7.69 (dt, 1H), 7.00 (dt, 1H), 6.55 (s, 1H), 4.56 (dd, 2H), 3.28-3.17 (m, 2H), 2.32-2.15 (m, 2H).

### Example V

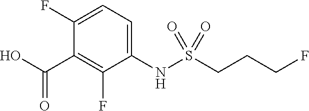

### 2,6-difluoro-3-(3-fluoropropylsulfonamido)benzoic acid

2,6-Difluoro-3-(N-(3-fluoropropylsulfonyl)propylsulfonamido)benzoate (38 g, 120 mmol) was dissolved in 5:2 THF/MeOH (250 mL), and a solution of lithium hydroxide (8.77 g, 366 mmol) in water (50 mL) was added. The reaction mixture was stirred at room temperature for four hours. The majority of the organic solvents were then removed in vacuo. 2.5N HCl (500 mL) was slowly added to the mixture, and the resulting solid was filtered and rinsed with cold ether to give 2,6-difluoro-3-(3-fluoropropylsulfonamido)benzoic acid as a solid (29.3 g, 81% yield). ¹H NMR (400 MHz, CDCl₃ ppm) 9.85 (s, 1H), 7.54 (dt, 1H), 7.21 (dt, 1H), 4.54 (td, 2H), 2.20-2.00 (m, 2H), 3.24-3.18 (m, 2H).

### Example W

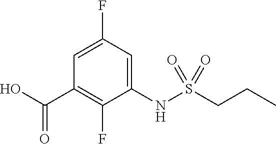

### 2,5-difluoro-3-(propylsulfonamido)benzoic acid

Step A: 2,5-Difluorobenzoic acid (2.01 g, 9.90 mmol, 31.3% yield) was dissolved in concentrated sulfuric acid (25 mL) and cooled to 0° C. Nitric Acid (1.46 mL, 34.8 mmol) was added, and the reaction mixture was stirred at room temperature for one hour. The solution was extracted with DCM (3×), and the combined organic layers were dried over sodium sulfate and concentrated. The residue was purified by column:chromatography (1:1 hexanes:1% HCOOH/EtOAc) giving 2,5-difluoro-3-nitrobenzoic acid (2.01 g, 31.3%) as a solid.

Step B: 2,5-Difluoro-3-nitrobenzoic acid (2.00 g, 9.847 mmol) was dissolved in MeOH (60 mL). TMSCl (6.220 mL, 49.24 mmol) was added, and the reaction mixture was stirred at reflux for 4 hours. The reaction mixture was concentrated to about 20 mL, and the crystals produced were filtered and dried under high vacuum providing methyl 2,5-difluoro-3-nitrobenzoate (1.55 g, 72.4%) as a crystalline solid.

Step C: Methyl 3-amino-2,5-difluorobenzoate (96.5%) was prepared according to the general procedure for Example B, Step B, substituting methyl 2,5-difluoro-3-nitrobenzoate for methyl 2,6-difluoro-3-nitrobenzoate.

Step D: Methyl 2,5-difluoro-3-(N-(propylsulfonyl)propylsulfonamido)benzoate was prepared according to the general procedure for Example B, Step C, substituting methyl 3-amino-2,5-difluorobenzoate for methyl 3-amino-2,6-difluorobenzoate.

Step E: 2,5-Difluoro-3-(propylsulfonamido)benzoic acid (83.8%, two steps) was prepared according to the general procedure for Example C substituting methyl 2,5-difluoro-3-(N-(propylsulfonyl)propylsulfonamido)benzoate for methyl 2,6-difluoro-3-(N-(propylsulfonyl)propylsulfonamido)benzoate. ¹H NMR (400 MHz, d₆-DMSO) δ 13.67 (br s, 1H), 10.07 (s, 1H), 7.46-7.50 (m, 1H), 7.38-7.42 (m, 1H), 3.17-3.21 (m, 2H), 1.70-1.76 (m, 2H), 0.95-0.99 (m, 3H); m/z (APC)-neg) M−1=278.1.

### Example X

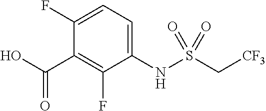

### 2,6-difluoro-3-(2,2,2-trifluoroethylsulfonamido)benzoic acid

Step A: 2,2,2-Trifluoroethyl-sulfonyl chloride (459 mL, 4.15 mmol) was slowly added to a solution of methyl 3-amino-2,6-difluorobenzoate (311 g, 1.66 mmol) and pyridine (0.806 mL, 9.97 mmol) in dichloromethane (8.92 mL, 139 mmol), while applying external cooling using an acetone dry ice bath. The reaction mixture was stirred for 45 minutes, and the dry ice bath was removed. The reaction mixture was kept stirring for another hour. The mixture was diluted with EtOAc (100 mL), washed with water (2×25 mL) and brine (25 mL), dried (Na₂SO₄), filtered, and then concentrated to an oil. The crude product was purified by column chromatography, eluting with 15% EtOAc/hexane to afford methyl 2,6-difluoro-3-(2-trifluoroethylsulfonamido) benzoate as a solid (513 mg, 92.6% yield). ¹H NMR (400 MHz, d₆-DMSO) δ 8.10-8.01 (m, 1H), 7.48 (t, 1H), 4.68 (s, 2H), 4.58 (s, 2H), 3.98 (s, 3H).

Step B: 2,6-Difluoro-3-(2-trifluoroethylsulfonamido)benzoic acid was prepared according to the general procedure for Example C, substituting methyl 2,6-difluoro-3-(2-trifluoroethylsulfonamido) benzoate for methyl 2,6-difluoro-3-(N-(propylsulfonyl)-propylsulfonamido)benzoate. ¹H NMR (500 MHz, d₆-DMSO) δ 14.08 (br s, 1H), 9.75 (s, 1H), 7.58-7.52 (m, 1H), 7.25 (t, 1H), 3.15-3.11 (s, 2H).

### Example Y

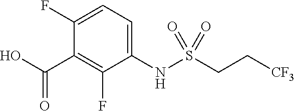

### 2,6-difluoro-3-(3,3,3-trifluoropropylsulfonamido)benzoic acid

Step A: Methyl 2,6-difluoro-3-(N-(3,3,3-trifluoropropylsulfonyl)-3,3,3-trifluoropropyl-sulfonamido) benzoate was made according to the general procedure for Example B, substituting 3,3,3-trifluoropropyl sulfonyl chloride for propane-1-sulfonyl chloride. ¹H NMR (400 MHz, d₆-DMSO) δ 8.05-7.99 (m, 1H), 7.44 (t, 1H), 4.62 (t, 2H), 4.50 (t, 2H), 3.93 (s, 3H), 3.89-3.74 (m, 4H), 2.26-2.11 (m, 4H).

Step B: 2,6-Difluoro-3-(3,3,3-trifluoropropylsulfonamido)benzoic acid was prepared according to the general procedure for Example C, substituting methyl 2,6-difluoro-3-(N-(3,3,3-trifluoropropylsulfonyl)-3,3,3-trifluoropropylsulfonamido)benzoate for methyl 2,6-difluoro-3-(N-(propylsulfonyl)-propylsulfonamido)benzoate. ¹H NMR (500 MHz, d₆-DMSO) δ 14.05 (br s, 1H), 9.71 (s, 1H), 7.56-7.50 (m, 1H), 7.20 (t, 1H), 3.12-3.08 (m, 2H), 1.73-1.66 (m, 2G).

### Example Z

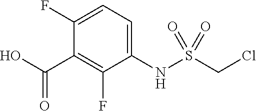

### 2,6-difluoro-3-(2-chloromethylsulfonamido)benzoic acid

Step A: Methyl 2,6-difluoro-3-(N-(2-chloromethylsulfonyl)-2-chloromethyl-sulfonamido) benzoate was made according to the general procedure for Example B, substituting 2-chloromethyl sulfonyl chloride for propane-1-sulfonyl chloride. ¹H NMR (400 MHz, d₆-DMSO) δ 8.08-7.97 (m, 1H), 7.45 (t, 1H), 4.65 (s, 2H), 4.55 (s, 2H), 4.02 (s, 3H).

Step B: 2,6-Difluoro-3-(2-chloromethylsulfonamido)benzoic acid was prepared according to the general procedure for Example C, substituting methyl 2,6-difluoro-3-(N-(2-chloromethylsulfonyl)-2-chloromethylsulfonamido)benzoate for methyl 2,6-difluoro-3-(N-(propylsulfonyl)-propylsulfonamido)benzoate. ¹H NMR (500 MHz, d₆-DMSO) δ 14.10 (br s, 1H), 9.78 (s, 1H), 7.62-7.56 (m, 1H), 7.28 (t, 1H), 3.19-3.15 (s, 2H).

### Example AB

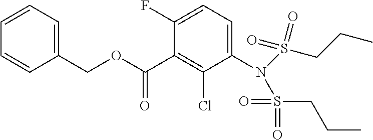

### benzyl 2-chloro-6-fluoro-3-(N-(propylsulfonyl)propylsulfonamido)benzoate

Step A: Benzyl 3-amino-2-chloro-6-fluorobenzoate (56%) was prepared according to the general procedure for Example K, substituting 2-chloro-4-fluoroaniline for 4-chloro-2-fluoroaniline. ¹H NMR (400 MHz, d₆-DMSO) δ 7.48-7.32 (m, 5H), 7.11-7.05 (t, 1H), 6.94-6.89 (q, 1H), 5.53-5.49 (s, 2H), 5.41-5.39 (s, 2H).

Step B: Benzyl 3-amino-2-chloro-6-fluorobenzoate (330 mg, 1.2 mmol) was dissolved in dry dichloromethane (11.8 mL). Triethylamine (0.494 mL, 3.54 mmol) was added, and the mixture was chilled to 0° C. Propane-1-sulfonyl chloride (0.332 mL, 2.95 mmol) was then added via syringe. Once the addition was complete, the mixture was allowed to warm to ambient temperature and stir for 16 hours. The mixture was diluted with dichloromethane (11 mL) and washed with water (2×50 mL) and brine (25 mL), dried over sodium sulfate, and concentrated. The resulting residue was applied directly to a silica gel column and eluted with a gradient (5% to 40%) of ethyl acetate-hexanes to provide benzyl 2-chloro-6-fluoro-3-(N-(propylsulfonyl)propylsulfonamido)benzoate (413 mg, 0.840 mmol, 71.1% yield). ¹H NMR (400 MHz, d₆-DMSO) δ 8.00-7.94 (q, 1H), 7.59-7.52 (t, 1H), 7.50-7.35 (m, 5H), 5.48-5.44 (s, 2H), 3.80-3.60 (m, 4H), 1.89-1.75 (m, 4H), 1.05-0.98 (t, 6H).

### Example AC

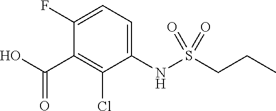

### 2-chloro-6-fluoro-3-(propylsulfonamido)benzoic acid

Step A: Benzyl 2-chloro-6-fluoro-3-(N-(propylsulfonyl)propylsulfonamido) benzoate (413.2 mg, 0.840 mmol) was dissolved in THF (8.4 mL) and 2.0M aqueous LiOH (1.26 mL). The mixture was refluxed for 16 hours and then allowed to cool to ambient temperature. The mixture was acidified to a pH of 0 with 1.0M HCl (5.0 mL) and then adjusted to a pH of 4 using saturated sodium bicarbonate. The mixture was extracted with EtOAc (2×). The extracts were washed with water (2×) and brine (1×), dried over sodium sulfate and concentrated to afford benzyl 2-chloro-6-fluoro-3-(propylsulfonamido)benzoate (174.5 mg, 0.4523 mmol, 53.9% yield). MS (APC)-neg) m/z=384.1 (M-H).

Step B: Benzyl 2-chloro-6-fluoro-3-(propylsulfonamido)benzoate (174.5 mg, 0.4523 mmol) was dissolved in 3:1 dioxane:water (7.5 mL) and treated with barium hydroxide (100.7 mg, 0.5879 mmol). The reaction mixture was heated to 80° C. for 16 hours and then allowed to cool to ambient temperature. The mixture was acidified to a pH of 0 with concentrated HCl. The reaction mixture was allowed to stir for 10 minutes, after which the pH was adjusted to a pH of 4 using saturated sodium bicarbonate. The mixture was extracted with EtOAc (2×). The extracts were washed with water (2×) and brine (1×), dried over sodium sulfate, and concentrated to afford 2-chloro-6-fluoro-3-(propylsulfonamido)benzoic acid (75.7 mg, 0.2560 mmol, 56.6% yield). MS (APC)-neg) m/z=293.9 (M-H).

### Example AD

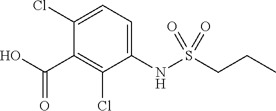

2,6-dichloro-3-(propylsulfonamido)benzoic acid

Step A: 2,6-Dichloro-3-nitrobenzoic acid (2.13 g, 9.03 mmol) was dissolved in 2:1 THF:saturated aqueous NH₄Cl and cooled to 0° C. The mixture was treated with zinc (11.8 g, 181 mmol). The reaction mixture was allowed to warm to ambient temperature and stir for 24 hours. The reaction mixture was filtered through GF/F paper while rinsing with THF. The mixture was acidified to a pH of 1 using 1.0M HCl and extracted with 15% 2-propanol:DCM (3×). The extracts were washed with water and brine, dried over sodium sulfate and concentrated to afford 3-amino-2,6-dichlorobenzoic acid (1.40 g, 6.82 mmol, 75.5% yield). MS (APC)-neg) m/z=203.6 (M-H).

Step B: 3-Amino-2,6-dichlorobenzoic acid (1.40 g, 6.82 mmol) was dissolved in dry dichloromethane (66.7 mL). Triethylamine (4.09 mL, 29.4 mmol) was added, and the mixture was chilled to 0° C. Propane-1-sulfonyl chloride (2.48 mL, 22 mmol) was then added via syringe. Once the addition was complete, the mixture was allowed to warm to ambient temperature and stir for 1 hour. The mixture was concentrated in vacuo and diluted with diethyl ether. The mixture was washed with 0.25M NaOH (80 mL), and the aqueous layer was acidified to a pH of 1 using 1.0M HCl. The aqueous layer was extracted with 15% 2-propanol:DCM (2×300 mL). The organic layer was collected, dried over sodium sulfate, and concentrated to afford 2,6-dichloro-3-(propylsulfonamido)benzoic acid (1.55 g, 4.96 mmol, 74.4% yield). ¹H NMR (400 MHz, d₆-DMSO) δ 9.77-9.75 (s, 1H), 7.84-7.80 (d, 1H), 7.71-7.68 (d, 1H), 3.82-3.72 (m, 2H), 1.89-1.70 (m, 2H), 1.05-1.03 (m, 3H).

### Example 1

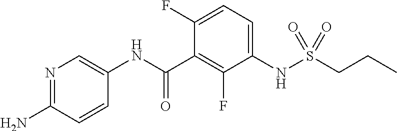

N-(6-aminopyridin-3-yl)-2,6-difluoro-3-(propylsulfonamido)benzamide

Diisopropylethylamine (0.266 mL, 1.53 mmol), 2,6-difluoro-3-(propylsulfonamido)benzoic acid (77 mg, 0.275 mmol), hydroxybenzotriazole monohydrate (46.7 mg, 0.305 mmol) and 1-ethyl-(3-dimethylaminopropyl)carbodiimide hydrochloride (59.6 mg, 0.336 mmol) were added to 2,5-diaminopyridine dihydrochloride (50 mg, 0.275 mmol) in dichloromethane (3 mL) and N,N-dimethylformamide (1 mL). The resulting mixture was stirred at ambient temperature. After 5 hours, the reaction mixture was diluted with brine and extracted twice with ethyl acetate. The combined ethyl acetate extracts were washed with brine, dried over magnesium sulfate, filtered, and evaporated to yield crude product (70 mg) as a film. This was chromatographed on a silica gel plug, eluting with 4:1 ethyl acetate:methanol. Fraction 2 contained solid (20.3 mg, yield 20%). ¹H NMR (400 MHz, CD₃OD) δ 8.17 (s, 1H), 7.79-7.75 (m, 1H), 7.66-7.59 (m, 1H), 7.10 (t, 1H), 6.62 (d, 1H), 3.09 (t, 2H), 1.91-1.81 (m, 2H), 1.05 (t, 3H); m/z (APCI pos) 371.1 (100%) [M+1].

### Example 2

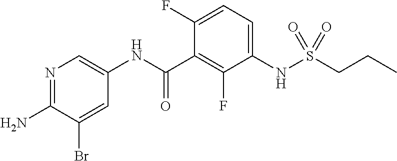

### N-(6-amino-5-bromopyridin-3-yl)-2,6-difluoro-3-(propylsulfonamido)benzamide

Prepared from 2,5-diamino-3-bromopyridine by the procedure of Example 1. Yield 20%; ¹H NMR (400 MHz, CDCl₃) δ 8.19 (d, 1H), 8.16 (br s, 1H), 8.03 (d, 1H), 7.01-6.96 (m, 1H), 5.19 (br s, 2H), 3.08-3.03 (m, 2H), 1.92-1.82 (m, 2H), 1.65 (br s, 1H), 1.03 (t, 3H); m/z (APCI pos) 371.1 (100%) [M+1].

### Example 3

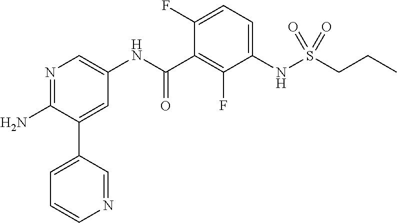

### N-(2-amino-3,3′-bipyridin-5-yl)-2,6-difluoro-3-(propylsulfonamido)benzamide

Sodium carbonate (33.2 mg, 0.313 mmol) was added to N-(6-amino-5-bromopyridin-3-yl)-2,6-difluoro-3-(propylsulfonamido)benzamide (46.9 mg, 0.104 mmol) in dimethoxyethane:ethanol:water (5:2:1; 7 mL) in a vial, and the mixture was sparged with nitrogen before PdCl₂(dppf)(DCM) catalyst (8.6 mg, 0.0104 mmol) was added. The mixture was stirred for 10 minutes, and then pyridyl-3-boronic acid (19.2 mg, 0.157 mmol) was added. The mixture was heated to 80° C. After 1.5 hours, the reaction mixture was filtered through celite and evaporated. The residue was partitioned between ethyl acetate and water. The organic layer was washed with brine, dried over magnesium sulfate, filtered, and evaporated to yield crude product as an oil. The crude was chromatographed on a Biotage silica gel column, eluting with 10:1 ethyl acetate:methanol. Fractions 8-12 contained a glassy product (16.2 mg, yield 35%). ¹H NMR (400 MHz, CDCl₃) δ 8.67-8.65 (m, 1H), 8.64-8.62 (m, 1H), 8.44 (br s, 1H), 8.23-8.22 (m, 1H), 7.91-7.83 (m, 2H), 7.68-7.62 (m, 1H), 7.44-7.40 (m, 1H), 7.00-6.95 (m, 1H), 4.77 (br s, 2H), 3.07-3.04 (m, 2H), 1.92-1.82 (m, 2H), 1.65 (br s, 1H), 1.03 (t, 3H); m/z (APCI pos) 448.1 (100%) [M+1].

### Example 4

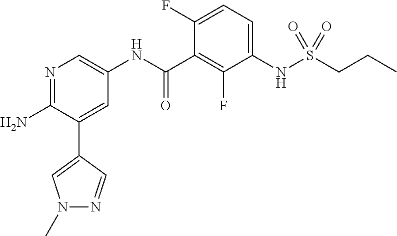

### N-(6-amino-5-(1-methyl-1H-pyrazol-4-yl)pyridin-3-yl)-2,6-difluoro-3-(propylsulfonamido)benzamide

Prepared from N-(6-amino-5-bromopyridin-3-yl)-2,6-difluoro-3-(propyl-sulfonamido)benzamide and 1-methylpyrazole-4-boronic acid pinacol ester by the procedure of Example 3. Yield 25%; ¹H NMR (400 MHz, CDCl₃) δ 8.20 (1, 1H), 8.06 (d, 1H), 7.87 (d, 1H), 7.69 (s, 1H), 7.68-7.61 (m, 2H), 6.98 (t, 1H), 4.86 (br s, 2H), 3.98 (s, 3H), 3.08-3.03 (m, 2H), 1.92-1.83 (m, 2H), 1.70 (br s, 1H), 1.03 (t, 3H); m/z (APCI neg) 449.2 (100%) [M−1].

### Example 5

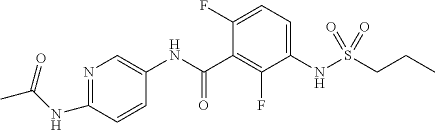

### N-(6-acetamidopyridin-3-yl)-2,6-difluoro-3-(propylsulfonamido)benzamide

Thionyl chloride (0.54 mL, 7.37 mmol) was added to 2,6-difluoro-3-(propylsulfonamido)benzoic acid (29.4 mg, 0.105 mmol) in toluene (2 mL). The mixture was heated to reflux. After 6 hours, the solution was evaporated, and a solid residue dried under high vacuum to afford crude 2,6-difluoro-3-(propylsulfonamido)benzoyl chloride. The crude 2,6-difluoro-3-(propylsulfonamido)benzoyl chloride was placed into chloroform (1 mL) to form a solution, and a solution of 2-acetamino-5-aminopyridine (15.9 mg, 0.105 mmol) and diisopropylethylamine (0.027 mL, 0.158 mmol) in chloroform (2 mL) and N,N-dimethylformamide (0.2 mL) was added. The mixture was refluxed overnight. The reaction mixture was evaporated and partitioned between ethyl acetate and water. The ethyl acetate was washed with saturated aqueous sodium bicarbonate, brine, dried over magnesium sulfate, filtered, and evaporated to yield a crude product as a solid (35 mg). The crude was chromatographed on a Biotage silica gel column with 10:1 dichloromethane:methanol. Fractions 6-9 contained product as a solid (13.6 mg, yield 31%). ¹H NMR (400 MHz, CD₃OD) δ 8.61 (s, 1H), 8.12-8.02 (m, 2H), 7.68-7.61 (m, 1H), 7.14-7.09 (m, 1H), 3.13-3.08 (m, 2H), 2.17 (s, 3H), 1.91-1.81 (m, 2H), 1.05 (t, 3H); m/z (APCI pos) 413.1 (100%) [M+1].

### Example 6

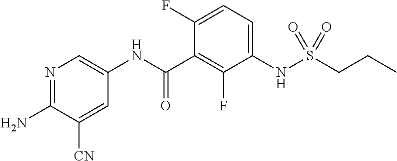

### N-6-amino-5-cyanopyridin-3-yl)-2,6-difluoro-3-(propylsulfonamido)benzamide

Cuprous cyanide (8.0 mg, 0.09 mmol) was added to a solution of N-(6-amino-5-bromopyridin-3-yl)-2,6-difluoro-3-(propylsulfonamido)benzamide (20 mg, 0.045 mmol) in N,N-dimethylformamide (0.455 mL). The mixture was heated in a Biotage Initiator microwave at 180° C. for 75 minutes. The mixture was diluted with ethyl acetate and washed twice with brine, dried over magnesium sulfate, filtered, and evaporated to yield a crude product. The crude was chromatographed on a Biotage silica gel column, eluting with 6:3:1 dichloromethane:acetonitrile:methanol. Fraction 2 contained product as a solid (4.4 mg, yield 25%). ¹H NMR (400 MHz, CD₃OD) δ 8.39 (br s, 1H), 8.18 (br s, 1H), 7.67-7.61 (m, 1H), 7.14-7.09 (m, 1H), 3.12-3.08 (m, 2H), 1.91-1.81 (m, 2H), 1.05 (t, 3H); m/z (APCI pos) 396.1 (100%) [M+1].

### Example 7

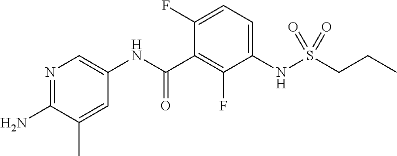

### N-(6-amino-5-methylpyridin-3-yl)-2,6-difluoro-3-(propylsulfonamido)benzamide

Step A: Ammonium chloride (73.4 mg, 1.37 mmol) and iron (583 mg, 10.4 mmol) were added to 3-methyl-5-nitropyridin-2-amine (200.0 mg, 1.31 mmol) in ethanol:water (4:1, 12.5 mL) in a round-bottom flask, and the mixture was heated at 80° C. for 3 hours. The reaction mixture was cooled down, and then filtered on a pad of celite. The filtrate was washed with a saturated solution of NaHCO₃. The organics were dried with sodium sulfate, filtered and concentrated in vacuo. The crude product was used directly in the next step.

Step B: Prepared from 3-methylpyridine-2,5-diamine by the procedure of Example 1. Yield 13%; ¹H NMR (500 MHz, DMSO-d₆) δ 11.06 (s, 1H), 9.82 (s, 1H), 8.38 (s, 1H), 7.76 (s, 1H), 7.65 (br s, 2H), 7.56 (q, 1H), 7.27 (t, 1H), 3.12 (t, 2H), 2.20 (s, 3H), 1.76 (m, 2H), 0.99 (t, 3H); m/z (ES-MS) 385.2 (100%) [M+1].

### Example 8

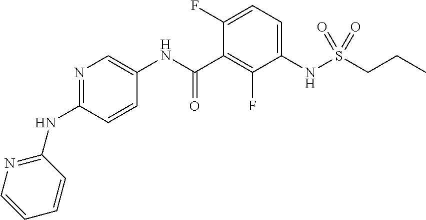

### 2,6-difluoro-3-(propylsulfonamido)-N-(6-(pyridin-2-ylamino)pyridin-3-yl)benzamide

Step A: A sealed microwave vial was charged with 2-bromopyridine (100 mg, 0.63 mmol), 2-amino-5-nitropyridine (115 mg, 0.82 mmol), tris(dibenzylideneacetone) dipalladium(0) (116 mg, 0.13 mmol), 1,3-bis(diphenylphosphino)propane (131 mg, 0.32 mmol), sodium tert-butoxide (104 mg, 1.08 mmol) and toluene (2.9 mL) and was then heated in a microwave reactor at 120° C. for 10 minutes. The reaction mixture was diluted with ethyl acetate and washed with a saturated solution of NaHCO₃. The organics were dried with sodium sulfate, filtered and concentrated in vacuo. The crude was purified by flash chromatography to afford 5-nitro-N-(pyridine-2-yl)pyridin-2-amine (148 mg, 53%).

Step B: A sealed microwave vial was charged with 5-nitro-N-(pyridin-2-yl)pyridin-2-amine (75.0 mg, 0.35 mmol), iron (232 mg, 4.16 mmol), ammonium chloride (74.2 mg, 1.39 mmol) and ethanol:water (4:1, 3.0 mL). The mixture was heated in a microwave reactor at 90° C. for 13 minutes. The reaction mixture was filtered on a pad of celite. The filtrate was diluted with ethyl acetate and washed with a saturated solution of NaHCO₃. The aqueous layer was extracted twice with ethyl acetate. The organics were dried with sodium sulfate, filtered and concentrated in vacuo. The crude was purified by flash chromatography to afford N2-(pyridin-2-yl)pyridine-2,5-diamine (45 mg, 35%).

Step C: Prepared from N2-(pyridin-2-yl)pyridine-2,5-diamine by the procedure of Example 5. Yield 57%; ¹H NMR (400 MHz, DMSO-d₆) δ 11.11 (s, 2H), 9.79 (s, 1H), 8.79 (s, 1H), 8.30 (d, 1H), 8.12 (d, 1H), 8.02 (br s, 1H), 7.57 (q, 1H), 7.47 (m, 2H), 7.28 (t, 1H), 7.16 (br s, 1H) 3.13 (t, 2H), 1.77 (m, 2H), 0.99 (t, 3H); m/z (ES-MS) 448.2 (100%) [M+1].

### Example 9

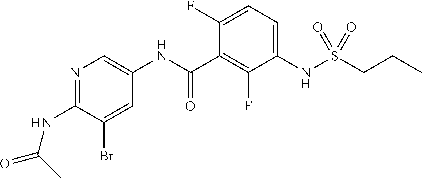

### N-(6-acetamido-5-bromopyridin-3-yl)-2,6-difluoro-3-(propylsulfonamido)benzamide

Step A: A mixture of 2-amino-3-bromo-5-nitropyridine (375 mg, 1.72 mmol) in acetic anhydride (5.5 mL) was heated at 80° C. for 2 hours. The reaction mixture was diluted with ethyl acetate and then washed with a saturated solution of NaHCO₃. The aqueous layer was extracted twice with ethyl acetate. The organics were dried with sodium sulfate, filtered and concentrated in vacuo. The crude was purified by flash chromatography to afford N-(3-bromo-5-nitropyridin-2-yl)acetamide (256 mg, 57%).

Step B: A sealed microwave vial was charged with N-(3-bromo-5-nitropyridin-2-yl)acetamide (100 mg, 0.38 mmol), iron (258 mg, 4.61 mmol), ammonium chloride (82.2 mg, 1.54 mmol) and ethanol:water (4:1, 3.3 mL). The mixture was heated in a microwave reactor at 90° C. for 13 minutes. The reaction mixture was filtered on a pad of celite. The filtrate was diluted with ethyl acetate and washed with a saturated solution of NaHCO₃. The aqueous layer was extracted twice with ethyl acetate. The organics were dried with sodium sulfate, filtered and concentrated in vacuo. The crude N-(5-amino-3-bromopyridin-2-yl)acetamide was directly used into the next step.

Step C: Prepared from N-(5-amino-3-bromopyridin-2-yl)acetamide by the procedure of Example 5. Yield 15%; ¹H NMR (400 MHz, DMSO-d₆) δ 11.27 (s, 1H), 10.11 (s, 1H), 9.80 (s, 1H), 8.62 (d, 1H), 8.47 (d, 1H), 7.61-7.55 (m, 1H), 7.28 (t, 1H), 3.14-3.10 (m, 2H), 2.03 (s, 3H), 1.76 (m, 2H), 0.99 (t, 3H); m/z (ES-MS) 493.1 (96.4%) [M+1].

### Example 10

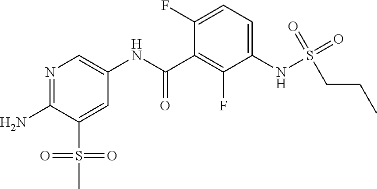

### N-(6-amino-5-(methylsulfonyl)pyridin-3-yl)-2,6-difluoro-3-(propylsulfonamido)benzamide

A sealed microwave vial was charged with N-(6-amino-5-bromopyridin-3-yl)-2,6-difluoro-3-(propylsulfonamido)benzamide (100 mg, 0.22 mmol), methanesulphinic acid sodium salt (36.9 mg, 0.29 mmol), sodium hydroxide (1.8 mg, 0.045 mmol), L-proline (5.1 mg, 0.045 mmol), copper iodide(I) (4.2 mg, 0.022 mmol) and dimethylsulfoxide (“DMSO”; 1.3 mL). The mixture was heated in a microwave reactor at 170° C. for 85 minutes. The reaction mixture was diluted with ethyl acetate and washed with a saturated solution of NaCl. The aqueous layer was extracted twice with ethyl acetate. The organics were dried with sodium sulfate, filtered and concentrated in vacuo. The crude product was purified by reverse phase HPLC to give N-(6-amino-5-(methylsulfonyl)pyridin-3-yl)-2,6-difluoro-3-(propylsulfonamido)benzamide (12 mg, yield 12%). ¹H NMR (400 MHz, DMSO-d₆) δ 10.90 (s, 1H), 9.76 (s, 1H), 8.49 (d, 1H), 8.33 (d, 1H), 7.57-7.51 (m, 1H), 7.25 (t, 1H), 6.64 (br s, 2H), 3.22 (s, 3H), 3.13-3.10 (m, 2H), 1.76 (m, 2H), 0.99 (t, 3H); m/z (ES-MS) 449.1 (96.8%) [M+1].

### Example 11

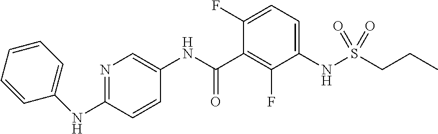

### 2,6-difluoro-N-(6-(phenylamino)pyridin-3-yl)-3-(propylsulfonamido)benzamide

Step A: A sealed microwave vial was charged with 2-bromo-5-nitropyridine (250 mg, 1.23 mmol), aniline (146 μL, 1.60 mmol), tris(dibenzylideneacetone)dipalladium(0) (113 mg, 0.12 mmol), 1,3-bis(diphenylphosphino)propane (127 mg, 0.31 mmol), sodium tert-butoxide (201 mg, 2.1 mmol) and toluene (4.5 mL). The mixture was heated in a microwave reactor at 120° C. for 10 minutes. The reaction mixture was diluted with ethyl acetate, and washed with a saturated solution of NaHCO₃. The organics were dried with sodium sulfate, filtered and concentrated in vacuo. The crude was purified by flash chromatography to afford 5-nitro-N-phenylpyridin-2-amine (91 mg, 34%).

Step B: A sealed microwave vial was charged with 5-nitro-N-phenylpyridin-2-amine (91.0 mg, 0.42 mmol), iron (283 mg, 5.07 mmol), ammonium chloride (90.5 mg, 1.69 mmol) and ethanol:water (4:1, 2.25 mL). The mixture was heated in a microwave reactor at 100° C. for 20 minutes. The reaction mixture was filtered on a pad of celite. The filtrate was diluted with ethyl acetate and washed with a saturated solution of NaHCO₃. The aqueous layer was extracted twice with ethyl acetate. The organics were dried with sodium sulfate, filtered and concentrated in vacuo. The crude N2-phenylpyridine-2,5-diamine was directly used into the next step.

Step C: Prepared from N2-phenylpyridine-2,5-diamine by the procedure of Example 5. Yield 8%; ¹H NMR (400 MHz, DMSO-d₆) δ 10.76 (s, 1H), 9.76 (s, 1H), 9.11 (br s, 1H), 8.42 (d, 1H), 7.91 (dd, 1H), 7.63 (d, 2H), 7.57-7.51 (m, 1H), 7.28-7.22 (m, 3H), 6.91-6.87 (m, 2H), 3.14-3.10 (m, 2H), 1.77 (m, 2H), 1.00 (t, 3H); m/z (ES-MS) 447.2 (100%) [M+1].

### Example 12

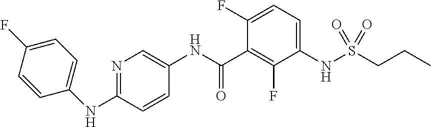

2,6-difluoro-N-(6-(4-fluorophenylamino)pyridin-3-yl)-3-(propylsulfonamido)benzamide

Step A: A sealed microwave vial was charged with 2-bromo-5-nitropyridine (250 mg, 1.23 mmol), 4-fluoroaniline (154 μL, 1.60 mmol), tris(dibenzylideneacetone) dipalladium(0) (113 mg, 0.12 mmol), 1,3-bis(diphenylphosphino)propane (127 mg, 0.31 mmol), sodium tert-butoxide (201 mg, 2.1 mmol) and toluene (4.5 mL). The mixture was then heated in a microwave reactor at 120° C. for 10 minutes. The reaction mixture was diluted with ethyl acetate and washed with a saturated solution of NaHCO₃. The organics were dried with sodium sulfate, filtered and concentrated in vacuo. The crude was purified by flash chromatography to afford N-(4-fluorophenyl)-5-nitropyridin-2-amine (98 mg, 34%).

Step B: A sealed microwave vial was charged with N-(4-fluorophenyl)-5-nitropyridin-2-amine (98.6 mg, 0.42 mmol), iron (283 mg, 5.07 mmol), ammonium chloride (90.5 mg, 1.69 mmol) and ethanol:water (4:1, 2.25 mL). The mixture was heated in a microwave reactor at 95° C. for 16 minutes. The reaction mixture was filtered on a pad of celite. The filtrate was diluted with ethyl acetate and washed with a saturated solution of NaHCO₃. The aqueous layer was extracted twice with ethyl acetate. The organics were dried with sodium sulfate, filtered and concentrated in vacuo. The crude N2-(4-fluorophenyl)pyridine-2,5-diamine was directly used into the next step.

Step C: Prepared from N2-(4-fluorophenyl)pyridine-2,5-diamine by the same procedure as in Example 3. Yield 24%; ¹H NMR (400 MHz, DMSO-d₆) δ 10.76 (s, 1H), 9.76 (s, 1H), 9.13 (br s, 1H), 8.40 (d, 1H), 7.91 (dd, 1H), 7.66-7.63 (m, 2H), 7.56-7.50 (m, 1H), 7.24 (t, 1H), 7.10 (t, 2H), 6.86 (d, 1H) 3.14-3.10 (m, 2H), 1.77 (m, 2H), 0.99 (t, 3H); m/z (ES-MS) 465.1 (100%) [M+1].

### Example 13

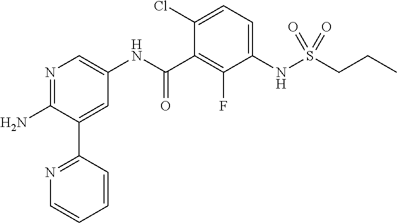

### N-(2′-amino-2,3′-bipyridin-5′-yl)-6-chloro-2-fluoro-3-(propylsulfonamido)benzamide

Step A: A solution of 3-bromopyridine-2,5-diamine (0.060 g, 0.32 mmol), 2-(4,4,5,5-tetramethyl-1,3,2-dioxaborolan-2-yl)pyridine (0.098 g, 0.48 mmol), NaOtBu (0.092 g, 0.96 mmol), diphenylphosphine oxide (0.019 g, 0.096 mmol) and Pd₂ dba₃ (0.015 g, 0.016 mmol) in p-dioxane (6 mL) was purged with Ar for 10 minutes and then heated to 110° C. under Ar for 16 hours. The reaction mixture was cooled to room temperature and concentrated. The residue was purified by column chromatography, eluting with hexanes/ethyl acetate (1:2), to give 2,3′-bipyridine-2′,5′-diamine (0.012 g, 20%). m/z (APC)-pos) M+1=187.3.

Step B: N-(2′-Amino-2,3′-bipyridin-5′-yl)-6-chloro-2-fluoro-3-(propylsulfonamido)benzamide was prepared according to Example 1, Step E using 2,3′-bipyridine-2′,5′-diamine and 6-chloro-2-fluoro-3-(propylsulfonamido)benzoic acid (0.015 g, 50%). ¹H NMR (400 MHz, MeOH-d₄) δ 8.7 (m, 1H), 8.3 (s, 1H), 8.2 (s, 1H), 7.9 (m, 1H), 7.8 (m, 1H), 7.6 (m, 1H), 7.4 (m, 2H), 3.1 (m, 2H), 1.9 (m, 2H), 1.1 (t, J=7.6 Hz, 3H); m/z (APC)-pos) M+1=464.2, 466.1.

### Example 14

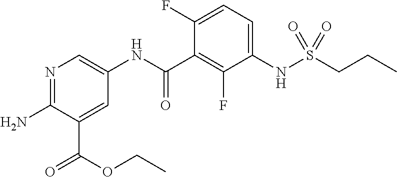

### ethyl 2-amino-5-(2,6-difluoro-3-(propylsulfonamido)benzamido)nicotinate

Step A: A 25 mL round bottom flask was charged with ethyl 2-amino-5-nitronicotinate (24 mg, 0.11 mmol; Collins, D. J. J. Chem. Soc. 1963, 1337-1339) and Pd/C (6.0 mg, 0.0057 mmol; 10 wt %). EtOH (10 mL) was added, and then H₂ gas was bubbled through the reaction mixture for 3 hours. The mixture was then filtered through a 0.45 micron PVDF frit (Acrodisc). The volatiles were removed to provide ethyl 2,5-diaminonicotinate as a solid (16 mg, 78% yield), which was used without further purification. m/z (APC)-pos) M+1=182.1.

Step B: Ethyl 2-amino-5-(2,6-difluoro-3-(propylsulfonamido)benzamido)-nicotinate (14%) was prepared according to the general procedure in Example 1, substituting ethyl 2,5-diaminonicotinate for 2,5-diaminopyridine dihydrochloride. m/z (APC)-pos) M+1=443.0.

### Example 15

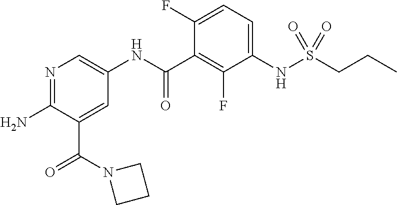

### N-(6-amino-5-(azetidine-1-carbonyl)pyridin-3-yl)-2,6-difluoro-3-(propylsulfonamido)benzamide

Step A: A solution of 2-aminonicotinic acid (1.01 g, 7.31 mmol) was dissolved in concentrated H₂SO₄ (ca 5 mL) and cooled to 0° C. Fuming nitric acid (0.4 mL, 8.6 mmol) was slowly added, and the reaction mixture was allowed to warm to room temperature. The mixture was then poured into ice water (100 mL). The resulting precipitates were filtered, washed with water (3×15 mL) and then Et₂O (3×10 mL) to afford 2-amino-5-nitronicotinic acid as a solid (1.06 g, 52% yield).

Step B: (2-Amino-5-nitropyridin-3-yl)(azetidin-1-yl)methanone (38%) was prepared according to the general procure in Example 1, substituting 2-amino-5-nitronicotinic acid for 2,5-diaminopyridine dihydrochloride and azetidine for 2,6-difluoro-3-(propylsulfonamido)benzoic acid.

Step C: Azetidin-1-yl(2,5-diaminopyridin-3-yl)methanone (100%) was prepared according to the general procedure in Example 14, Step A, substituting (2-amino-5-nitropyridin-3-yl)(azetidin-1-yl)methanone for ethyl 2-amino-5-nitronicotinate.

Step D: N-(6-Amino-5-(azetidine-1-carbonyl)pyridin-3-yl)-2,6-difluoro-3-(propylsulfonamido)benzamide (16%) was prepared according to the general procedure in Example 1, substituting azetidin-1-yl(2,5-diaminopyridin-3-yl)methanone for 2,5-diaminopyridine dihydrochloride. m/z (APC)-pos) M+1=454.1.

The following compounds in Table 1 were prepared following the above procedures.

TABLE 1

| Ex. # | Structure | Name | MS/NMR |
|---|---|---|---|
| 16 | 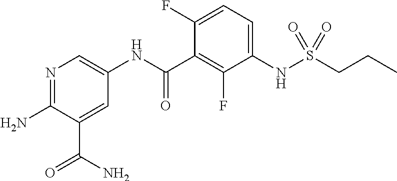 | 2-amino-5-(2,6- difluoro-3- (propylsulfonamido) benzamido)nicotinamide | m/z (APCI-pos) M + 1 = 412.1 |
| 17 | 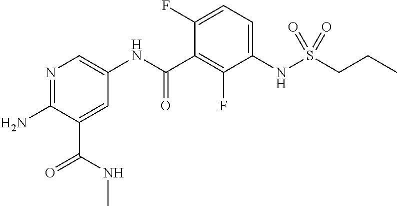 | 2-amino-5-(2,6- difluoro-3- (propylsulfonamido) benzamido)-N- methylnicotinamide | m/z (APCI-pos) M + 1 = 428.1 |
| 18 | 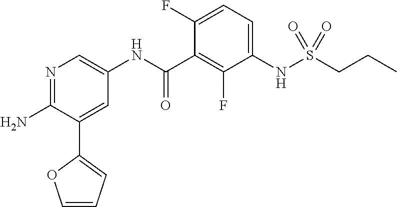 | N-(6-amino-5-(furan- 2-yl)pyridin-3-yl)- 2,6-difluoro-3- (propylsulfonamido) benzamide | m/z (APCI-pos) M + 1 = 437.1 |
| 19 | 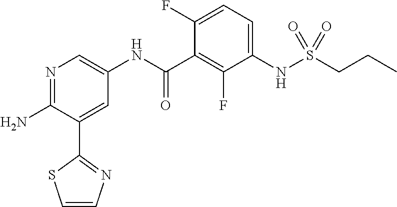 | N-(6-amino-5- (thiazol-2-yl)pyridin- 3-yl)-2,6-difluoro-3- (propylsulfonamido) benzamide | m/z (APCI-pos) M + 1 = 454.1 |

While the invention has been described in conjunction with the enumerated embodiments, it will be understood that they are not intended to limit the invention to those embodiments. On the contrary, the invention is intended to cover all alternatives, modifications and equivalents, which may be included within the scope of the present invention as defined by the claims. Thus, the foregoing description is considered as illustrative only of the principles of the invention.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound selected from Formula I: 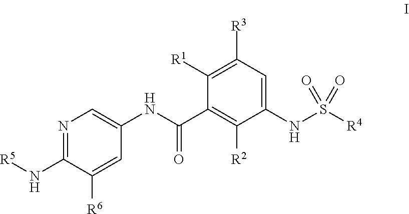
and stereoisomers, tautomers and pharmaceutically acceptable salts thereof, wherein:
R¹ and R² are independently selected from hydrogen, halogen, CN, C₁-C₃ alkyl and C₁-C₃ alkoxy;
R⁴ is C₃-C₅ cycloalkyl, C₁-C₆ alkyl, C₂-C₆ alkenyl, C₂-C₆ alkynyl, phenyl, a 5-6 membered heteroaryl, or NR^{b}R^{c}, wherein the cycloalkyl, alkyl, alkenyl, alkynyl and phenyl are optionally substituted with OR^{a}, halogen, phenyl, C₃-C₄ cycloalkyl or C₁-C₄ alkyl optionally substituted with halogen;
R⁵ is hydrogen, —C(═O)(C₁-C₄ alkyl), phenyl optionally substituted with halogen or C₁-C₄ alkyl, or a 5-6 membered heteroaryl;
R⁶ is hydrogen, halogen, CN, —SO₂(C₁-C₄ alkyl), C₁-C₄ alkyl, —C(═O)R^{d} or a 5-6 membered heteroaryl optionally substituted with C₁-C₄ alkyl;
each R^{a} is hydrogen or C₁-C₄ alkyl;
each R^{b} and R^{c} are independently selected from hydrogen and C₁-C₅ alkyl optionally substituted with halogen, or
R^{b} and R^{c} together with the nitrogen to which they are attached form a 4 to 6 membered heterocyclic ring;
R^{d} is —O(C₁-C₆ alkyl), NR^{e}R^{f} or a 4 membered heterocycle; and
each R^{e} and R^{f} are independently selected from hydrogen and C₁-C₆ alkyl.

2. A compound selected from Formula I: 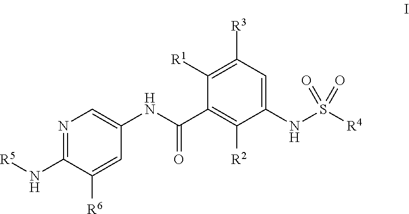
and stereoisomers, tautomers and pharmaceutically acceptable salts thereof, wherein:
R¹ and R² are independently selected from hydrogen, halogen, CN, C₁-C₃ alkyl and C₁-C₃ alkoxy;
R³ is hydrogen, halogen or C₁-C₃ alkyl;
R⁴ is C₃-C₅ cycloalkyl, C₁-C₆ alkyl, C₁-C₆ alkenyl, or C₁-C₆ alkynyl, wherein the cycloalkyl, alkyl, alkenyl and alkynyl are optionally substituted with OR^{a}, halogen or C₃-C₄ cycloalkyl;
R⁵ is hydrogen, —C(═O)(C₁-C₄ alkyl), phenyl optionally substituted with halogen or C₁-C₄ alkyl, or a 5 or 6 membered heteroaryl;
R⁶ is hydrogen, halogen, CN, —SO₂(C₁-C₄ alkyl), C₁-C₄ alkyl, or a 5-6 membered heteroaryl optionally substituted with C₁-C₄ alkyl; and
R^{a} is hydrogen or C₁-C₄ alkyl.

3. A compound of claim 1, wherein:
R¹, R² and R³ are independently selected from hydrogen, halogen or C₁-C₃ alkyl;
R⁴ is C₃-C₄ cycloalkyl, or C₁-C₆ alkyl optionally substituted with halogen, OH or C₃-C₄ cycloalkyl;
R⁵ is hydrogen, —C(═O)(C₁-C₄ alkyl), phenyl optionally substituted with halogen or C₁-C₄ alkyl, or a 5 or 6 membered heteroaryl; and
R⁶ is hydrogen, halogen, CN, —SO₂(C₁-C₄ alkyl), C₁-C₄ alkyl, or a 5-6 membered heteroaryl optionally substituted with C₁-C₄ alkyl.

4. A compound as claimed in any one of claims 1 to 3, wherein R¹, R² and R³ are independently selected from hydrogen, halogen or C₁-C₃ alkyl.

5. A compound as claimed in any one of claims 1 to 4, wherein the residue: 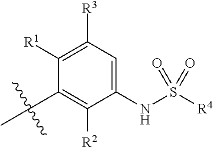
of Formula I, wherein the wavy line represents the point of attachment of the residue in Formula I, is selected from:

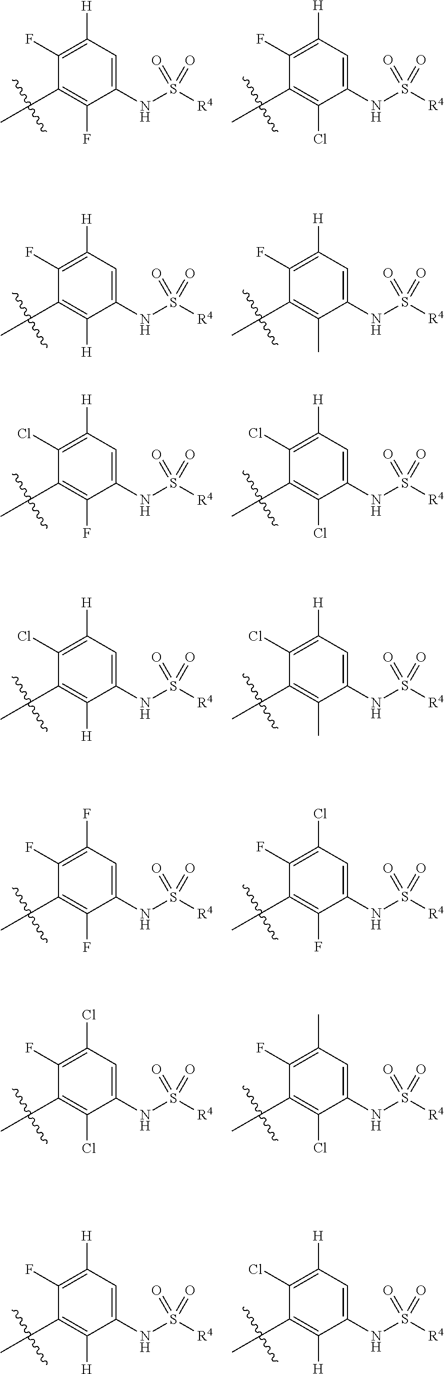

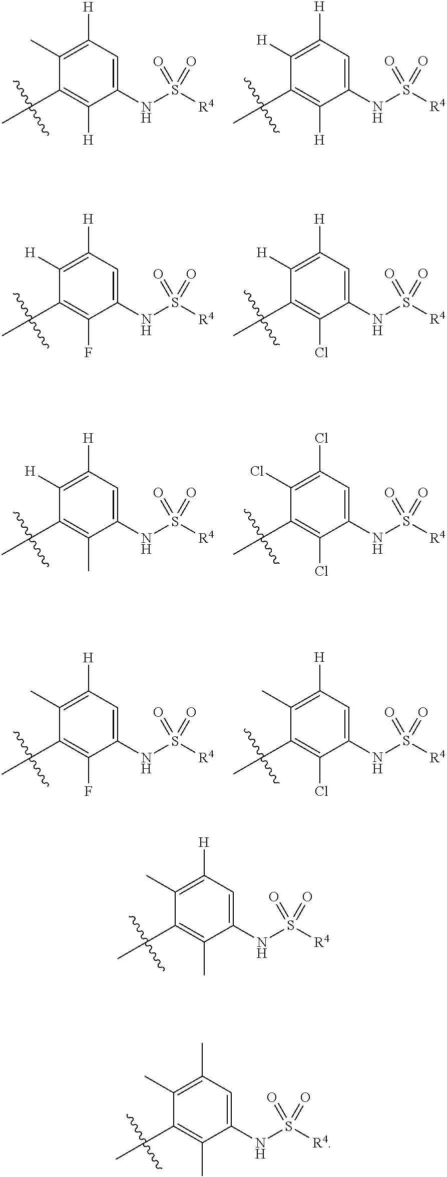

6. A compound as claimed in any one of claims 1 to 5, wherein R¹ and R² are F and R³ is hydrogen.

7. A compound as claimed in any one of claims 1 to 5, wherein R¹, R² and R³ are F.

8. A compound as claimed in any one of claims 1 to 5, wherein R¹ is F and R² is Cl and R³ is hydrogen.

9. A compound as claimed in any one of claims 1 to 5, wherein R¹ is Cl and R² is F and R³ is hydrogen.

10. A compound as claimed in any one of claims 1 to 5, wherein R¹ is F and R² is methyl and R³ is hydrogen.

11. A compound as claimed in any one of claims 1 to 5, wherein R¹ is methyl and R² is F and R³ is hydrogen.

12. A compound as claimed in any one of claims 1 to 5, wherein R¹ is F and R² and R³ are hydrogen.

13. A compound as claimed in any one of claims 1 to 5, wherein R¹ is Cl and R² and R³ are hydrogen.

14. A compound as claimed in any one of claims 1 to 5, wherein R² is F and R¹ and R³ are hydrogen.

15. A compound as claimed in any one of claims 1 to 14, wherein R⁴ is propyl, butyl, isobutyl, —CH₂CH₂CH₂F, —CH₂CH₂CF₃ or cyclopropylmethyl.

16. A compound as claimed in any one of claims 1 to 15, wherein R⁴ is propyl.

17. A compound as claimed in any one of claims 1 to 14, wherein R⁴ is —CF₃, —CH₂CF₃, —CH₂CH₂CH₂F, —CH₂CH₂CF₃, —CF₂CF₃ or —CF₂CF₂CF₃.

18. A compound of claim 1, wherein R⁴ is cyclopropyl, ethyl, propyl, butyl, isobutyl, —CH₂CH₂CH₂OH, —CH₂Cl, —CH₂CF₃, —CH₂CH₂CH₂F, —CH₂CH₂CF₃, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl, —NHCH₂CH₃, —NHCH₂CH₂CH₃, —N(CH₃)CH₂CH₃, —NHCH(CH₃)₂, —NHCH₂CHF₂, —N(CH₃)₂ or pyrrolidin-1-yl.

19. A compound of claim 1, wherein R⁴ is cyclopropyl, ethyl, propyl, isobutyl, —CH₂CH₂CH₂OH, —CH₂CH₂CH₂F, phenylmethyl, cyclopropylmethyl, phenyl, 2-fluorophenyl, 3-fluorophenyl, 4-fluorophenyl, 2,5-difluorophenyl, 4-chloro-3-trifluoromethylphenyl, 1-methyl-1H-imidazol-4-yl, furan-2-yl, pyridin-2-yl, pyridin-3-yl, thiophen-2-yl or —NHCH₂CH₃.

20. A compound as claimed in any one of claims 1 to 19, wherein R⁶ is hydrogen.

21. A compound as claimed in any one of claims 1 to 19, wherein R⁶ is halogen or CN.

22. A compound as claimed in any one of claims 1 to 19, wherein R⁶ is —SO₂(C₁-C₄ alkyl)

23. A compound as claimed in any one of claims 1 to 19, wherein R⁶ is C₁-C₄ alkyl

24. A compound as claimed in any one of claims 1 to 19, wherein R⁶ is a 5-6 membered heteroaryl selected from pyridinyl and pyrazolyl.

25. A compound as claimed in any one of claim 1 or 19, wherein R⁶ is selected from hydrogen, halogen, CN, —SO₂CH₃, methyl, pyridin-3-yl and 1-methyl-1H-pyrazol-4-yl.

26. A compound of claim 1, wherein R⁶ is selected from hydrogen, halogen, CN, —SO₂CH₃, methyl, —C(═O)CH₂CH₃, —C(═O)(azetidin-1-yl), —C(═O)NH₂, —C(═O)NHCH₃, pyridin-3-yl, pyridin-2-yl, 1-methyl-1H-pyrazol-4-yl, furan-2-yl and thiazol-2-yl.

27. A compound as claimed in any one of claims 1 to 26, wherein R⁵ is hydrogen.

28. A compound as claimed in any one of claims 1 to 26, wherein R⁵ is —C(═O)(C₁-C₄ alkyl).

29. A compound as claimed in any one of claims 1 to 26, wherein R⁵ is phenyl optionally substituted with halogen or C₁-C₄ alkyl.

30. A compound as claimed in any one of claims 1 to 26, wherein R⁵ is a 5-6 membered heteroaryl, wherein the heteroaryl is pyridinyl.

31. A compound as claimed in any one of claims 1 to 26, wherein R⁵ is selected from hydrogen, —C(═O)CH₃, phenyl, 4-fluorophenyl and pyridin-2-yl.

32. A compound of Formula I as defined in any one of claim 1 or 2 and named in any one of Examples 1 to 12 herein.

33. A compound of Formula I as defined in claim 1 and named in any one of Examples 13 to 19 herein.

34. A pharmaceutical composition, comprising a compound as claimed in any one of claims 1 to 33, and a pharmaceutically acceptable carrier or excipient.

35. A method of preventing or treating a disease or disorder modulated by b-Raf, comprising administering to a mammal in need of such treatment an effective amount of a compound of any one of claims 1 to 33.

36. A method of preventing or treating cancer, comprising administering to a mammal in need of such treatment an effective amount of a compound of any one of claims 1 to 33, alone or in combination with one or more additional compounds having anti-cancer properties.

37. The method of claim 36, wherein the cancer is a sarcoma.

38. The method of claim 36, wherein the cancer is a carcinoma.

39. The method of claim 38, wherein the carcinoma is squamous cell carcinoma.

40. The method of claim 36, wherein the carcinoma is adenoma or adenocarcinoma.

41. The method of claim 36, wherein the cancer is breast, ovary, cervix, prostate, testis, genitourinary tract, esophagus, larynx, glioblastoma, neuroblastoma, stomach, skin, keratoacanthoma, lung, epidermoid carcinoma, large cell carcinoma, NSCLC, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, hairy cells, buccal cavity and pharynx (oral), lip, tongue, mouth, pharynx, small intestine, colon-rectum, large intestine, rectum, brain and central nervous system, Hodgkin's and leukemia.

42. A method of treating a hyperproliferative disease in a mammal comprising administering a therapeutically effective amount of a compound of any one of claims 1 to 33 to the mammal.

43. A compound as claimed in any one of claims 1 to 33 for use in therapy.

44. A compound as claimed in any one of claims 1 to 33 for use in the treatment of a hyperproliferative disease.

45. Use of a compound of any one of claims 1 to 33 in the manufacture of a medicament for the treatment of a hyperproliferative disease.

46. Use of a compound as claimed in any one of claims 1 to 33, in the manufacture of a medicament, for use as a b-Raf inhibitor in the treatment of a patient undergoing cancer therapy.

47. A method of preventing or treating kidney disease, comprising administering to a mammal in need of such treatment an effective amount of a compound of any one of claims 1 to 33, or a stereoisomer, tautomer or pharmaceutically acceptable salt thereof, alone or in combination with one or more additional compounds.

48. The method of claim 47, wherein the kidney disease is polycystic kidney disease.

49. A compound of any one of claims 1 to 33 for use in the treatment of a kidney disease.

50. The compound of claim 49, wherein the kidney disease is polycystic kidney disease.

51. Use of a compound of any one of claims 1 to 33 in the manufacture of a medicament for the treatment of a kidney disease.

52. The use of claim 51, wherein the kidney disease is polycystic kidney disease.

53. A pharmaceutical composition comprising a compound as claimed in any one of claims 1 to 33 for use in the treatment of a hyperproliferative disease.

54. A pharmaceutical composition comprising a compound as claimed in any one of claims 1 to 33 for use in the treatment of cancer.

55. A pharmaceutical composition comprising a compound as claimed in any one of claims 1 to 33 for use in the treatment of kidney disease.

56. The composition of claim 55, wherein the kidney disease is polycystic kidney disease.

57. A compound selected from Formula III: 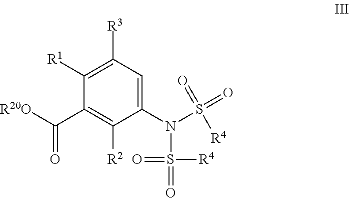
wherein R²⁰ is hydrogen, C₁-C₆ alkyl, benzyl or phenyl;
R¹ and R² are independently selected from hydrogen, halogen, CN, C₁-C₃ alkyl and C₁-C₃ alkoxy;
R³ is hydrogen, halogen or C₁-C₃ alkyl;
R⁴ is C₃-C₅ cycloalkyl, C₁-C₆ alkyl, C₁-C₆ alkenyl, or C₁-C₆ alkynyl, wherein the cycloalkyl, alkyl, alkenyl and alkynyl are optionally substituted with OR^{a}, halogen or C₃-C₄ cycloalkyl; and
R^{a} is hydrogen or C₁-C₄ alkyl.

58. A compound of claim 44, wherein R¹, R² and R³ are independently selected from hydrogen, halogen or C₁-C₃ alkyl; and
R⁴ is C₃-C₄ cycloalkyl or C₁-C₆ alkyl optionally substituted with OH, halogen or C₃-C₄ cycloalkyl.

59. A process for preparing compounds of Formula I, comprising:
(a) coupling a compound of Formula 1: 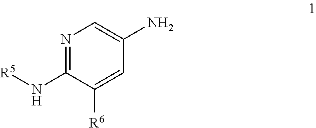
wherein R⁵ is hydrogen, —C(═O)(C₁-C₄ alkyl), phenyl optionally substituted with halogen or C₁-C₄ alkyl, or a 5 or 6 membered heteroaryl; and R⁶ is hydrogen, halogen, CN, —SO₂(C₁-C₄ alkyl), C₁-C₄ alkyl, or a 5-6 membered heteroaryl optionally substituted with C₁-C₄ alkyl;
with a compound of Formula 2: 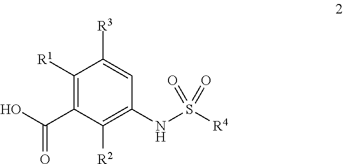
wherein R¹ and R² are independently selected from hydrogen, halogen, CN, C₁-C₃ alkyl and C₁-C₃ alkoxy; R³ is hydrogen, halogen or C₁-C₃ alkyl; R⁴ is C₃-C₅ cycloalkyl, C₁-C₆ alkyl, C₁-C₆ alkenyl, or C₁-C₆ alkynyl, wherein the cycloalkyl, alkyl, alkenyl and alkynyl are optionally substituted with OR^{a}, halogen or C₃-C₄ cycloalkyl; and R^{a} is hydrogen or C₁-C₄ alkyl;
to provide a compound of Formula I: 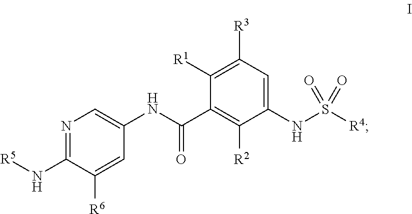
(b) coupling a compound of Formula 1: 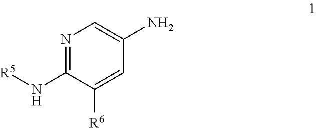
wherein R⁵ is hydrogen, —C(═O)(C₁-C₄ alkyl), phenyl optionally substituted with halogen or C₁-C₄ alkyl, or a 5 or 6 membered heteroaryl; and R⁶ is hydrogen, halogen, CN, —SO₂(C₁-C₄ alkyl), C₁-C₄ alkyl, or a 5-6 membered heteroaryl optionally substituted with C₁-C₄ alkyl;
with a compound of Formula 4: 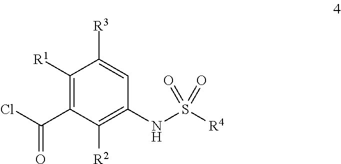
wherein R¹ and R² are independently selected from hydrogen, halogen, CN, C₁-C₃ alkyl and C₁-C₃ alkoxy; R³ is hydrogen, halogen or C₁-C₃ alkyl; and R⁴ is C₃-C₅ cycloalkyl, C₁-C₆ alkyl, C₁-C₆ alkenyl, or C₁-C₆ alkynyl, wherein the cycloalkyl, alkyl, alkenyl and alkynyl are optionally substituted with OR^{a}, halogen or C₃-C₄ cycloalkyl; and R^{a} is hydrogen or C₁-C₄ alkyl;
to provide a compound of Formula I: 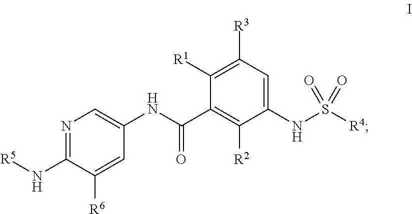
(c) coupling a compound of Formula 1: 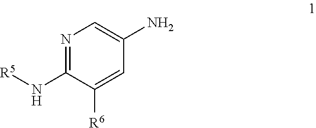
wherein R⁵ is hydrogen, —C(═O)(C₁-C₄ alkyl), phenyl optionally substituted with halogen or C₁-C₄ alkyl, or a 5 or 6 membered heteroaryl; and R⁶ is hydrogen, halogen, CN, —SO₂(C₁-C₄ alkyl), C₁-C₄ alkyl, or a 5-6 membered heteroaryl optionally substituted with C₁-C₄ alkyl;
with a compound of Formula III: 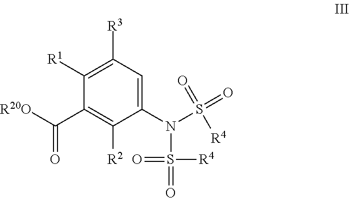
wherein R²⁰ is hydrogen, C₁-C₆ alkyl, benzyl or phenyl; R¹ and R² are independently selected from hydrogen, halogen, CN, C₁-C₃ alkyl and C₁-C₃ alkoxy; R³ is hydrogen, halogen or C₁-C₃ alkyl; R⁴ is C₃-C₅ cycloalkyl, C₁-C₆ alkyl, C₁-C₆ alkenyl, or C₁-C₆ alkynyl, wherein the cycloalkyl, alkyl, alkenyl and alkynyl are optionally substituted with OR^{a}, halogen or C₃-C₄ cycloalkyl; and R^{a} is hydrogen or C₁-C₄ alkyl
to provide a compound of Formula 6: 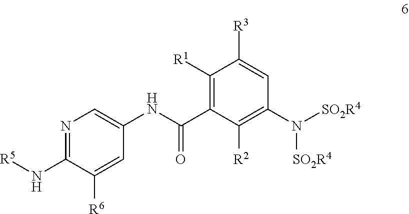
followed by hydrolysis to provide a compound of Formula I: 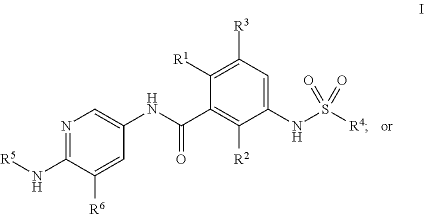
(d) coupling a compound of Formula 7: 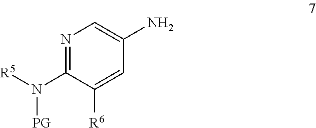
wherein PG is an amine protecting group; R⁵ is hydrogen, —C(═O)(C₁-C₄ alkyl), phenyl optionally substituted with halogen or C₁-C₄ alkyl, or a 5 or 6 membered heteroaryl; and R⁶ is hydrogen, halogen, CN, —SO₂(C₁-C₄ alkyl), C₁-C₄ alkyl, or a 5-6 membered heteroaryl optionally substituted with C₁-C₄ alkyl;
with a compound of Formula III: 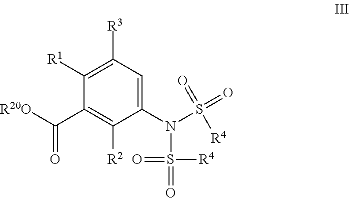
wherein R²⁰ is hydrogen, C₁-C₆ alkyl, benzyl or phenyl; R¹ and R² are independently selected from hydrogen, halogen, CN, C₁-C₃ alkyl and C₁-C₃ alkoxy; R³ is hydrogen, halogen or C₁-C₃ alkyl; R⁴ is C₃-C₅ cycloalkyl, C₁-C₆ alkyl, C₁-C₆ alkenyl, or C₁-C₆ alkynyl, wherein the cycloalkyl, alkyl, alkenyl and alkynyl are optionally substituted with OR^{a}, halogen or C₃-C₄ cycloalkyl; and R^{a} is hydrogen or C₁-C₄ alkyl;
to provide a compound of Formula 9: 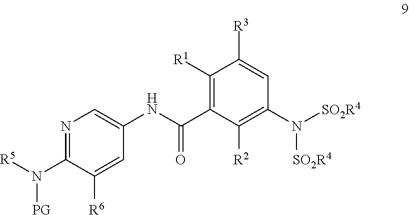
followed by hydrolysis and deprotection to provide a compound of Formula I: [chemical structure]
